(19) United States
(12) Patent Application Publication
(10) Pub. No.: US 2016/0022151 A1
(43) Pub. Date: Jan. 28, 2016

(54) NONINVASIVE METHOD OF MEASURING CARDIOVASCULAR PARAMETERS, MODELLING THE PERIPHERAL VASCULAR LOOP, ANALYZING VASCULAR STATUS, AND IMPROVING CARDIOVASCULAR DISEASE DIAGNOSIS

(71) Applicant: Charles L. DAVIS, Vancouver, WA (US)

(72) Inventor: Charles L. DAVIS, Vancouver, WA (US)

(21) Appl. No.: 14/776,520

(22) PCT Filed: Mar. 15, 2014
(86) PCT No.: PCT/US2014/030047

§ 371 Date: Sep. 14, 2015

Related U.S. Application Data
(60) Provisional application No. 61/786,539, filed on Mar. 15, 2013.

(51) Int. Cl. A61B 5/022; A61B 5/02; A61B 5/00

(52) U.S. Cl. CPC A61B 5/022; A61B 5/7278; A61B 5/02007; A61B 5/725; A61B 5/4875

(57) ABSTRACT

Methods of obtaining physiological parameters of a body fluid compartment. One method identifying a transitional relationship of the depletion body fluid volume indication values and the replenished body fluid volume indication values referenced to a series of pressure values, thereby indicating the physiological parameter of the body fluid compartment

### CROSS-REFERENCE TO RELATED APPLICATION

The present application claims priority to co-pending U.S. Provisional Application No. 61/786,539 filed on 15 Mar. 2013, incorporated herein by reference.

### BACKGROUND OF THE INVENTION

1. Field of the Invention

The present invention relates to a method of noninvasive measurement of cardiovascular parameters in a subject, data analysis methods for determining the status of the peripheral vascular bed, and improvements in cardiovascular disease diagnosis.

2. Discussion of the Related Art

The present invention is based upon the methods of noninvasive measurement disclosed in patent number U.S. Pat. No. 6,749,567 and U.S. Pat. No. 7,118,534 which are included in their entirety herein by reference.

In U.S. Pat. No. 6,749,567 the inventor disclosed methods of measuring the structure and organization of the Peripheral Vascular Loop by identifying state changes in the Volume vs Pressure data obtained from a coextensively applied pressure applicator and volume indicator for the purpose of determining physiologic parameters. Key parameters that were identified and claimed in that disclosure were Segmentation of the vasculature along the pathway of blood flow by inflections in the slope of volume versus pressure, Central Venous Pressure (CVP), Arterial Blood Pressure (ABP), and Compliance values of the various segments of the Peripheral Vascular Loop (PVL). The present invention will disclose further improvements on that methodology which will improve vascular disease diagnosis, treatment, and prevention.

Prior to the disclosures made in U.S. Pat. Nos. 6,749,567 and 7,118,534, noninvasive cardiovascular monitoring methods had been based upon pulsatile data and associated vascular models such as the Windkessel model. Many prior art disclosures made use of the Windkessel model for derivation of cardiovascular parameters such as vascular compliance, peripheral vascular resistance, and cardiac output. However, the vascular pulsations are generally damped out or reflected prior to reaching the capillary beds and therefore have little use in diagnosing the state of the veins and capillaries in the overall structure of the PVL. It is clinically important to determine the status of the veins and capillaries in relationship to the status of the arteries in order to understand the organizational structure of the cardiovascular system. The so called Capacitive Vessels have effectively been a hidden and unmonitored region of the PVL. Ultrasound has been available for observing and measuring attributes of individual veins but no means have been available for measuring the relative fluid compliance status of the capacitive vessels, capillaries, and arteries in relationship to each other. Means have been available for general hydration analysis of a patient but means have not been available for differentiating in what fluid compartments of the body the fluid is being stored? Body composition monitoring methods claim to be able to differentiate between intracellular water and extracellular water but it does not relate specifically to the cardiovascular system structure. New methods will be disclosed for making these determinations and developing new models of the cardiovascular system for improved diagnosis and treatment of cardiovascular disease.

In U.S. Pat. No. 6,749,567 the inventors disclosed methods of measuring the structure and organization of the Peripheral Vascular Loop by identifying state changes in the Volume vs Pressure data obtained from a coextensively applied pressure applicator and volume indicator for the purpose of determining physiologic parameters. Slope of Volume was an indicator identified for determination of CVP and segmentation of the vasculature. Key parameters that were identified and claimed in that disclosure were Central Venous Pressure (CVP), Arterial Blood Pressure (ABP), and Compliance values of the different segments of the Peripheral Vascular System. A key element of U.S. Pat. No. 6,749,567 was the recognition of state changes (Inflection Pressures) along the pathway of flow denoted by changes in slope of the volume relative to pressure. The present invention will disclose further improvements in identifying state changes which will further improve noninvasive cardiovascular modelling and disease diagnosis. Many of these new disclosures involve the interpretation of the relationship of associated state changes in the Depletion versus the Replenishment fluid data. The present invention will also disclose improved vascular models that will help physicians, researchers, and physiologists to better understand, diagnose, and treat cardiovascular disease.

Methods will be disclosed regarding determination of new physiologic parameters and improvements in the determination of existing physiologic parameters; improvements in segmentation methodologies and identification of Inflection Pressures; improved methods for determination of arterial blood pressure and noninvasive measurement of CVP values. Methods for noninvasive measurement of new parameters such as Stressed (V_{S}) and Unstressed (V_{US}) Volumes by vascular segment will be disclosed.

### BACKGROUND

U.S. Pat. No. 6,749,567 disclosed a method of measuring the pulsatile pressures and volumes as well as the residual (nonpulsatile) vascular volumes and pressures in sequential serial segments of the Circulatory System in both pulsating and non-pulsating vessels of the body. The U.S. Pat. No. 6,749,567 inventors disclosed methods for segmenting the vasculature by state changes in the volume vs pressure data that was acquired by coextensive relationship between the pressure application device and the volume measurement device. Elements of the circulatory system were identified in U.S. Pat. No. 6,749,567 as being either Pulsatile or Residual in character and behavior. Particular parameters of the circulatory system were identified and claimed in U.S. Pat. No. 6,749,567 including Central Venous Pressure (CVP), vessel compliance, static fluid pressure, blood oxygen level, adjacent fluid compartments, diastolic blood pressure of the large arteries, mean blood pressure of the large arteries, systolic blood pressure of the large arteries, static fluid pressure of the nonvascular fluid compartment, and a fluid volume of the body fluid compartments. New disclosures in the present invention will reveal the structure of the Peripheral Vascular Loop (PVL) and additional characteristics of the PVL that reveal the physiologic control mechanisms of the cardiovascular (CV) system for controlling blood flow in the body. The methods disclosed for data acquisition in U.S. Pat. No. 6,749,567 are crucial in revealing these new relationship elements of the PVL within the CV system and their importance to optimization of fluids and medications in the management of diseases such as Heart Failure, Hypertension, and Shock. Clinical benefit can be realized by knowing in which physiologic compartment residual fluid is being stored.

Further U.S. Pat. No. 7,118,534 discloses an additional method for noninvasive measurement of physiologic fluid parameters such as CVP. The present invention will disclose methods for determining improved and additional physiologic parameters and disease diagnosis by the methods similar to those disclosed in U.S. Pat. Nos. 6,749,567 and 7,118,534 to further benefit healthcare delivery, and disease diagnosis and treatment.

### SUMMARY

The present invention relates to a method of noninvasive measurement of cardiovascular parameters in a subject, data analysis methods for determining the status of the peripheral vascular bed, and improvements in cardiovascular disease diagnosis. In particular the present invention relates to a method of noninvasive measurement of cardiovascular parameters associated with the invention disclosed in U.S. Pat. No. 6,749,567 including;
1. Vascular Models, PVL and Vascular Compliance Stack
2. Relative (relational diagnosis of disease) values of compliance, resistance and pressure are indicative of disease diagnosis
3. Filtering methods for vascular information extraction from multi-source data
4. Elastance is not Resistance but may be proportional to Resistance
5. Improved methods for determining CVP
6. Hysteresis, lead or lag determination
7. Determination of Stressed vs Unstressed volume by segment
8. Determination of hardness of the vessel.
9. Relationship of CVP to venous segments as means of diagnosing vascular disease
10. Relationship of Mean arterial pressure as means of diagnosing vascular disease and hydration status
11. Means for determining vascular Decompensation
12. Methods for finding Inflection Pressures

### BRIEF DESCRIPTION OF THE DRAWINGS

For a better understanding of the invention, and to show how the same may be carried into effect, reference will now be made, by way of example, to the accompanying drawings, in which elements of this invention are demonstrated.

FIG. 1—Is a schematic representation of the Peripheral Vascular Loop illustrating the major vessel types, branching of vessels, physical laws governing flow, Inflection Pressures, blind spots, and prior art limitations.

FIG. 2—Is an illustration of the individual Peripheral Vascular Loop with one pathway of flow, the individual vessel types in the PVL, the four quadrants of the PVL, The roles of each quadrant of the PVL in promoting blood flow, and the varying effects of vessel Compliance relative to the type of vessel.

FIG. 3a—Is an illustration of the various kinds of PVL's that exist in the body overall.

FIG. 3b—Is a schematic of the pressure and flow relationships of several PVL's in the body thereby relating the PVL's from the arms to the Splanchnic bed.

FIG. 4a—is a drawing of a representative blood vessel segment without regard for any specific type, which identifies the forces found in the functional behaviors of a blood vessel.

FIG. 4b—is a drawing of an end view of a blood vessel expressing forces within the vessel lumen, outside the vessel, and within the wall of the vessel. It also shows a drawing of the interrelationship of the cuff and the bioimpedance sensor in making measurements of the vasculature.

FIG. 4c—is a diagram of the elements of a vessel and the application of Laplace's Law.

FIG. 4d—is a diagram of the sensor assembly used to collect pressure and volume data.

FIGS. 5a-5c—are diagrams of three different Vascular Compliance Stacks from three different people all having the same blood pressures.

FIG. 6—Cardiovascular control system interactions

FIG. 7a—is a graph of Vascular Elastance Profile (from animal study) for Depletion and Replenishment illustrating the ‘Venous Elastance’ which is the driving force behind venous return.

FIG. 7b—is a graph of Vascular Elastance Profile (human) for Depletion and Replenishment illustrating the ‘Venous Elastance’ which is the driving force behind venous return.

FIG. 8—is a graph demonstrating the relationship between the cuff pressure gradient and the bioimpedance changes measured by the sensor.

FIGS. 9a-9b—are two graphical examples of human PVL's illustrating the Residual Volume characteristics versus pressure for both Depletion and Replenishment.

FIG. 10a—is an example of the correlation function for Depletion and Replenishment data taken on a well compensated animal vasculature prior to blood loss.

FIG. 10b—is an example of the correlation function profile for Depletion and Replenishment data taken on a De-compensated animal vasculature following blood loss.

FIG. 11—is a graph of filtered vessel compliance profile for Depletion and Replenishment data

FIG. 12—is a graph of the correlation function profile which is scaled up to show the low pressure minimas for both Depletion and Replenishment data which are an alternative method for determining CVP.

FIG. 13—is a graph of Slope of Volume data demonstrating relationship between the Depletion and Replenishment data relative to identification of CVP. This graph also illustrates the “roaming” characteristic of CVP relative to the various venous segment boundaries.

FIG. 14a—is a graph that illustrates the frequency characteristics of residual fluid volumes in the vasculature and means for identifying the inflection pressures of individual segments across the PVL spectrum from Depletion data.

FIG. 14b—is a graph that illustrates the frequency characteristics of residual fluid volumes in the vasculature and means for identifying the inflection pressures of individual segments across the PVL spectrum from Replenishment data.

FIG. 14c—is a graph that illustrates the Very Low Frequency Band Volume Analysis from 0.2 to 0.6 Hz and the means for identifying inflection pressures at Filling Pressures and Mean Pressures of the vessel segment using Depletion data.

FIG. 14d—is a graph that illustrates the Very Low Frequency Band Volume Analysis from 0.2 to 0.6 Hz and the means for identifying inflection pressures at Filling Pressures and Mean Pressures of the vessel segment using Replenishment data.

FIG. 15a—is a graph that illustrates determination of Stressed Volume and Unstressed Volume by Vascular Segment using Replenishment data.

FIG. 15b—is a graph that illustrates determination of Stressed Volume and Unstressed Volume by Vascular Segment using Depletion data.

FIG. 16a—Deleted

FIG. 16b—is a graph that illustrates bleed and infusion cycles during the animal trial.

FIG. 16c—is a graph that illustrates Swan Ganz Catheter Derived Cardiac Output and Systemic Vascular Resistance during the animal trial.

FIG. 16d—is a graph that illustrates Vascular (De-)Compensation Function during the animal trial.

FIG. 16e—is a graph that illustrates Central Venous Pressure (CVP) during the animal trial.

FIG. 17—is a table of Vascular State.

FIG. 18—is a schematic of an Electrical Model of the PVL.

FIG. 19a—Deleted.

FIG. 19b—Deleted.

FIG. 20a—is a graph of a first reading of bleed test during the animal trial.

FIG. 20b—is a graph of a second reading of bleed test during the animal trial.

FIG. 20c—is a graph of a fifteenth reading of bleed test during the animal trial.

FIG. 20d—is a graph of a seventeenth reading of bleed test during the animal trial.

### DETAILED DESCRIPTION

### Terminology

“Transition Points”, “state transitions”, and “state changes”, are used synonymously to indicate a change from one pressure state (indicative of a particular fluid pressure and hence a particular vessel type according to aspects of the present invention) to a different pressure state.

“Known pressure value” as used herein includes known or measured values at the time of force application.

“Linear relationship” as used herein includes curvilinear relationships for ease and simplicity of explanation, unless otherwise noted.

“Graphing” as used herein includes any physical or virtual representation or construct referencing one type of value against another type of value, unless otherwise noted.

“Upstream” is used herein in the sense of an area of higher fluid pressure, while “downstream” indicates an area of lower fluid pressure.

“Residual Fluids” are static non time varying fluid volumes in compartments of the body

“Pulsatile Fluids” are time varying fluid volumes in compartments of the body

“Vascular Resistance” is the resistance to the flow of blood in the vasculature

“Filling Pressure” is the pressure that fills the vessel segment. It is the highest pressure associated with any particular vessel segment.

“Vascular Compliance” is the attribute of the vessel segment defined by ΔV/ΔP with ΔV being the change in residual volume of the segment and ΔP being the change in transmural pressure across the wall of the vessel

“Vascular Elastance” is the attribute of the vessel segment defined by ΔP/ΔV with ΔV being the change in residual volume of the segment and ΔP being the change in transmural pressure across the wall of the vessel. Elastance is the inverse of Compliance.

“Unstressed Volume” is the volume that it takes to fill the vessel without causing any transmural pressure greater than zero.

“Stressed Volume” is the volume in the vessel that causes the transmural pressure to be greater than zero.

“Homeostasis” is the balance of forces existing in the PVL structure.

Disease is the errant organization of the PVL forces creating an imbalance that impedes flow.

### Vascular Models;

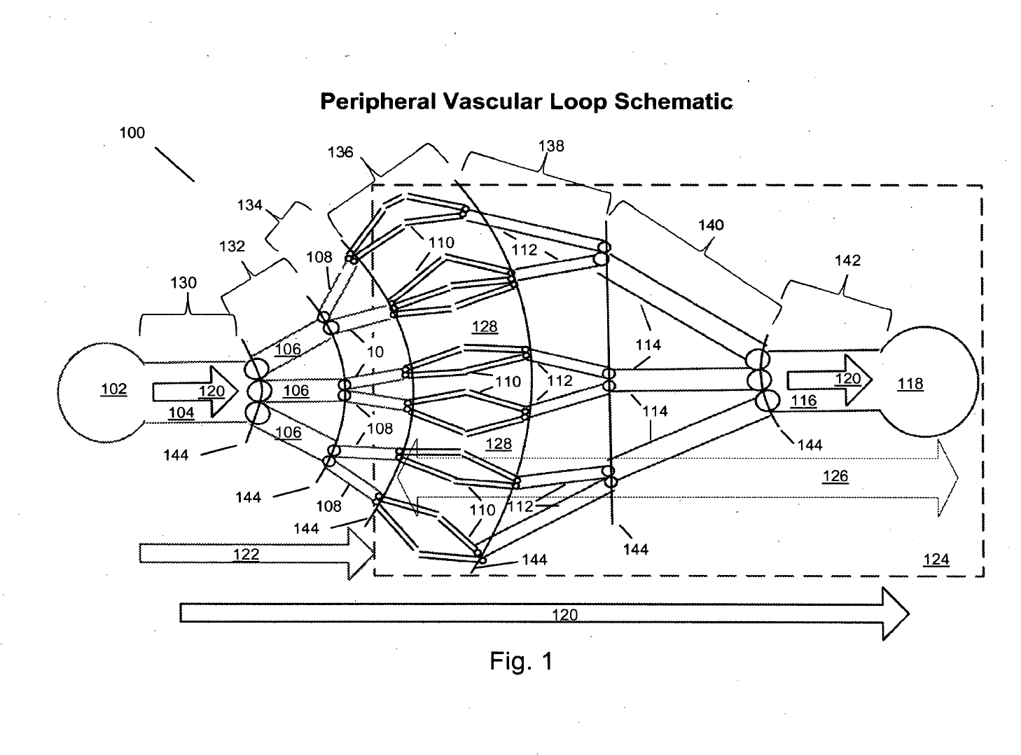

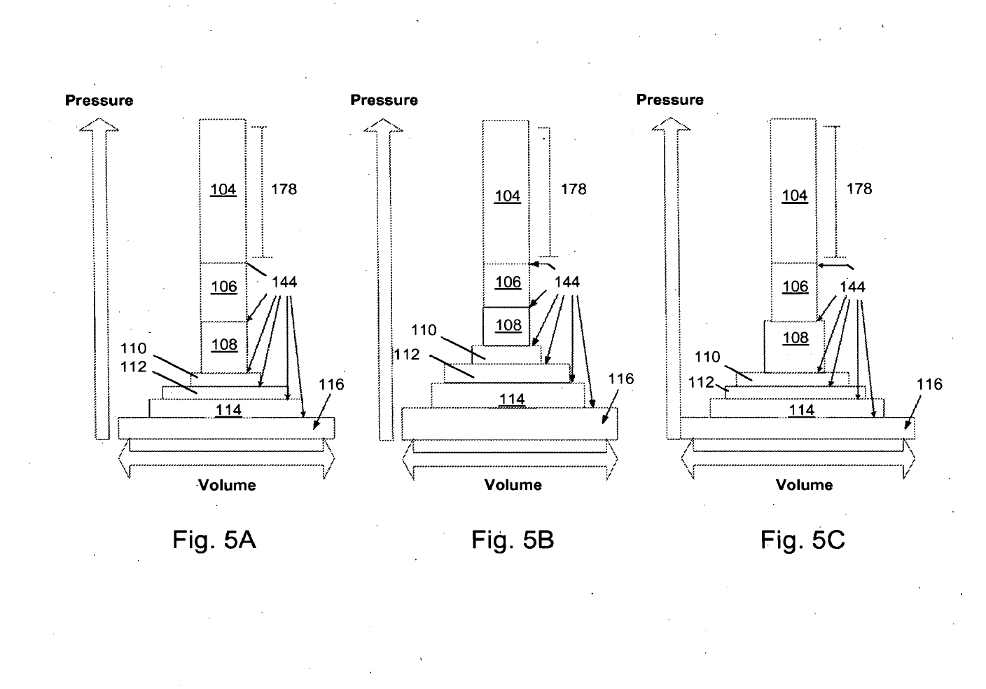

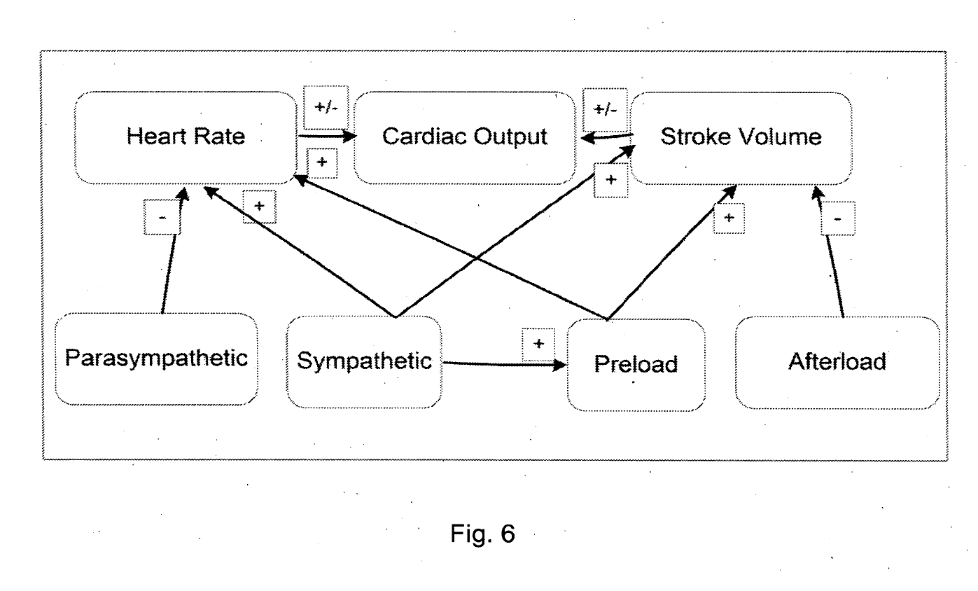

It is commonly known that transport of fluids within the body of a subject is accomplished by the cardiovascular system, as illustrated in FIGS. 1, 2 and 3. What is not commonly known is the organizational structure of the vascular bed and how that structure relates to disease diagnosis and management. An important feature of the present invention is the recognition that the physiologic control mechanisms of blood flow (neurological, endocrine, and renal), actively manipulate the organizational structure of the Volume vs Pressure (Compliance) characteristics of the vascular bed in a local region of the body. These physiologic control manipulations affect the compliance status (ΔV/ΔP) of the vessel walls of blood vessels at each stage or segment of the PVL in order to produce what the inventors call “the stacked or sequential compliance model” (SCM) of the vasculature (FIG. 5). The inventors recognized that the nature of blood flow in the body is serial and that there were key ‘Inflection Pressures’ along the pathway of flow which segmented the blood stream into compartments of residual fluid volume defined by the Compliance of that particular segment. FIG. 5 shows three different organizational structures of the SCM with each structure producing equivalent arterial blood pressures (ABP) and central venous pressure (CVP) but having different blood flow characteristics. Blood flow is clearly the primary purpose of the cardiovascular system. From this example it can be seen that ABP and CVP are not the only determinates of blood flow. It will be demonstrated in this disclosure how the relative compliance organization of the SCM is a fundamental indicator of vascular disease state. The propulsion of fluids through the cardiovascular system is accomplished by pulsatile pressurization of a network of blood vessels called Arteries by the periodic contraction of the heart. Blood flows from the heart through several stages or segments of arteries with each stage branching into smaller arteries in the direction of flow. The three stages of arteries are called Large Arteries, Small Arteries, and Arterioles. The arterioles are the third and final artery stage which branch into Capillaries. It is believed that the arterioles contribute the most to Peripheral Vascular Resistance (PVR) of all the arteries.

The Capillaries are a unique type of vessel which facilitates the exchange of gases and nutrients from the blood into the cells of the body. Capillaries are the smallest of all the vessels in the PVL. An important observation is that every cell of the body is physically located within 2 or 3 cells of a Capillary in order for this exchange to take place. Therefore, for proper respiration and feeding of the cells anywhere in the body, the physical structure that feeds the capillaries, i.e. Arterioles, Small Arteries, and Large Arteries must exist in a region of the body represented by a physical slice of a limb.

After the Capillaries the blood drains into the Veins, flowing first into the Venules, that then flow into the Small Veins, that then flow into the Large Veins and return to the heart. As the applied pressure of the pressure applicator increases against the coextensive volume sensor, the mobile volume in the PVL under the measurement region of the pressure applicator/volume sensor assembly, begins to decrease as the cuff pressure overcomes the physiologic pressure of the lowest pressure region of the PVL. Each vascular segment depletes volume as the cuff pressure continues to climb with segments collapsing in order of their internal physiologic pressurization. This affords the opportunity to noninvasively determine the organizational structure of the PVL.

### Introduction to the Peripheral Vascular Loop (PVL) (FIG. 1)

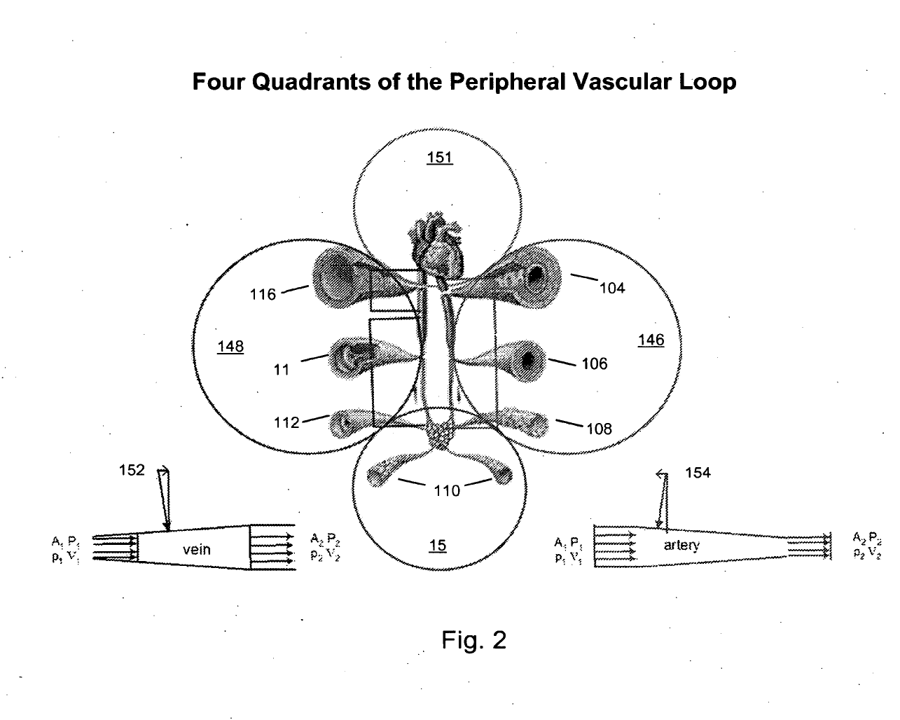

To understand the invention, one has to first understand the Peripheral Vascular Loop (PVL) in FIGS. 1 and 2 which illustrates some of the limitations of prior cardiovascular measurement methods and models. The PVL is a comprehensive model of the vascular system which accounts for both the pulsatile and the residual fluid volume behaviors of all the segments of the PVL.

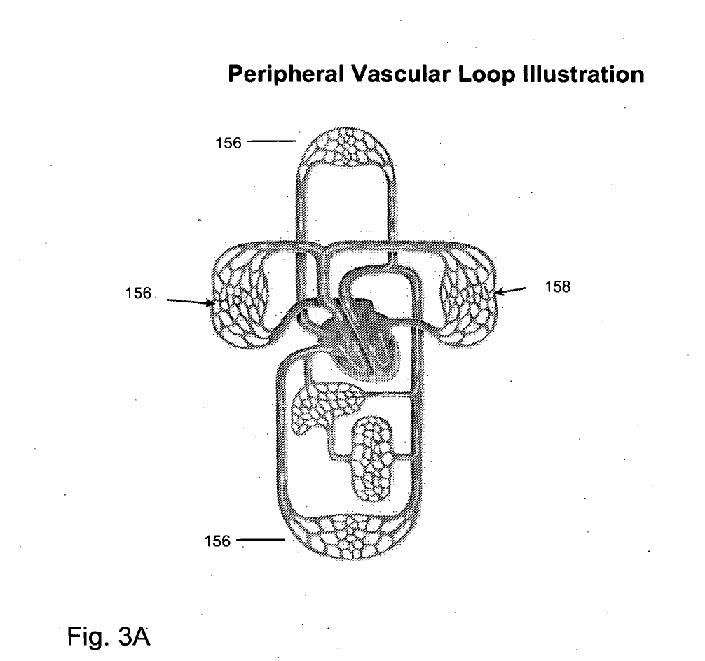

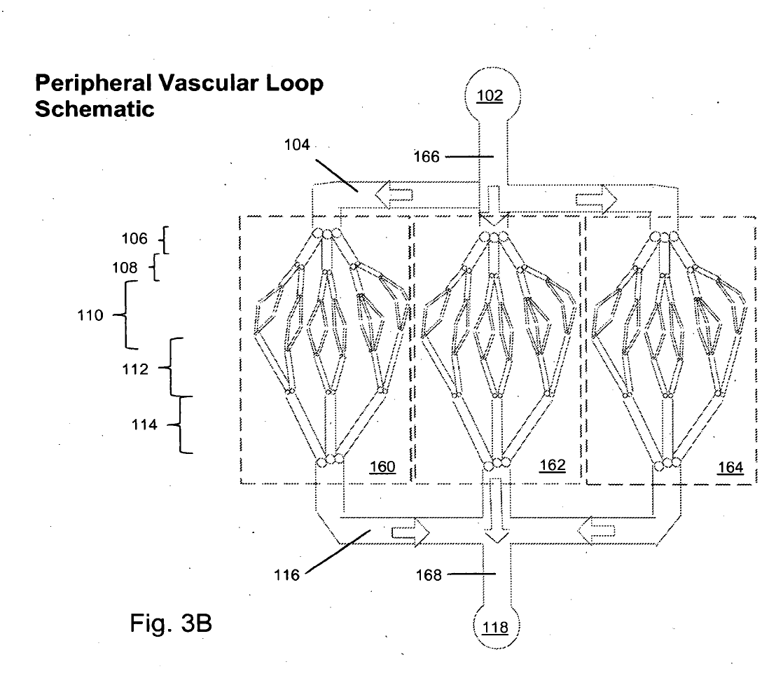

Blood flows around the Peripheral Vascular Loop (PVL) according to the compliance status of sequential segments of the PVL. The PVL spans vessels from the left side of the heart through the microvascular bed to the right side of the heart following the natural flow of blood (FIGS. 1 and 2). The Invention is an innovative noninvasive peripheral vascular analysis instrument because it measures the Volumes and Pressures in all segments of the PVL. From the Volume and Pressure data, the Invention calculates the Segment Boundaries for all PVL segments. The invention furthermore identifies relative features of the individual PVL structure by comparing Inflection Pressures during the depletion cycle to the inflection pressures obtained from the replenishment cycle. These relative features represent improvements to the art over U.S. Pat. No. 6,749,567. The PVL segments are functionally associated with the anatomic segments of the peripheral vascular bed commonly referred to in physiologic textbooks as the Large Arteries, Small Arteries, Arterioles, Capillaries, Venules, Small Veins, and Large Veins. The Peripheral Vascular Loop is a model derived from the perspective of blood flow through the vascular bed from the left side of the heart, through the arteries, through the capillaries, and through the veins back to the right heart and the anatomical vessel types that are often referenced in physiology and anatomy text books that comprise this structure in the body. In general there are three segments of the PVL, Arteries, Capillaries, and Veins. Furthermore, there are three stages of Arteries, Large Arteries, Small Arteries and Arterioles and there are three stages of Veins, Venules, Small Veins, and Large Veins. The physical structure of the PVL is as shown in FIGS. 1 and 3 with the large arteries branching into Small Arteries which then branch into Arterioles. The Arterioles branch into Capillaries where the biochemical work of the cardiovascular system is performed through the exchange of oxygen and nutrients with the cells of the body. The reduced blood flow leaves the Capillaries on the venous end and merges into Venules which then merge into Small Veins which then merge into Large Veins on the blood's trip back to the heart. An objective of this patent is to disclose that these sequential segments of the PVL have their own unique physiologic attributes such as Compliance, Resistance, and Pressure which effect Blood Flow in the PVL. U.S. Pat. No. 6,749,567 discloses the physiologic background and data acquisition methods used by this invention. This invention relates to useful physiologic information that is extracted from the basic Volume and Pressure data described in U.S. Pat. No. 6,749,567 and additional methods of relating to this data for useful physiologic monitoring.

### The Peripheral Vascular Loop (PVL) and Control of Venous Return

The Peripheral Vascular Loop (PVL) is comprised of 3 of the 4 quadrants of the cardiovascular system, FIG. 2. It is a network of blood vessels which represent a pathway for blood to flow from the left heart through several levels of arteries to a capillary and then through several levels of veins in order to return to the right heart. This network must exist in a slice of the limb since every cell of that limb exists within 2 to 3 cells of a capillary. This is an amazing architecture but we know it must exist or else the cells of that limb would die and we know they do not die, but live. In FIG. 2 we can see that blood leaves the left heart and flows into a network of arteries that are in effect tapered down in the direction of flow. This effective downward taper offers resistance to flow because of the Elastance of the Arteries and produces arterial blood pressure (ABP). This is the blood pressure that we commonly measure at the physician's office. It is a pulsatile pressure that is represented by three values, Systolic (the highest value), Diastolic (the lowest value), and Mean (the mathematical mean value of the pressure waveform). In current medical practice, the arterial pressures are the only pressure values that are commonly measured to assess the viability and wellbeing of the cardiovascular system. Arterial pressures are regulated and controlled by the myriad of physiologic control loops so the underlying PVL structure could be undergoing dramatic changes without affecting arterial blood pressure. There are three levels of arteries involved in the flow of blood to the capillary beds. What are the roles played by the three stages of arteries in regulating ABP? What special roles in the regulation of blood flow are played by each of the three levels of arteries?

Once the blood has crossed the capillaries, it now becomes venous blood. It is important to recognize that the venous taper in the direction of flow is opposite to that found in the arteries. The venous taper goes from small to large in the direction of flow while the arterial taper goes from large to small. This mechanical feature of the PVL promotes venous return and may be the dominant factor contributing to ‘Preload’ of the heart. There has long been controversy surrounding the definitions of ‘Preload’ and the sources of Preload as it relates to overall cardiovascular performance indicated by cardiac output [38]. Current clinical concerns regarding ‘fluid responsiveness’ relate directly to the limited understanding of what contributes to Preload and how normal physiology controls Preload versus how abnormal physiology adversely affects Preload. The simple answer is that Preload involves more vascular physiology than fluid volume! It is also important to understand the difference between ‘Residual Fluid Volume’ and its component parts ‘Stressed’ and ‘Unstressed’ volumes in the vascular segments. In particular it is these relative characteristics in the Veins that contribute substantially to the Preload on the heart. The relative amounts of Stressed vs Unstressed volumes in the veins is determined by the active physiologic control of the Wall Tension (Elastance) or Compliance of the veins. These control mechanisms are generally Autonomic Nervous System (ANS) and biochemical mediators that affect the amount of ‘Squeeze Force’ that the veins are exerting on the blood volume in order to return it to the heart. The so called ‘Venous Pump’ is often described as a function of skeletal muscle operating on the veins in order to squeeze blood back towards the heart. Venous valves are known to be functional elements of a weaker forcing function that regulates venous return. Therefore, it is not just the amount of blood volume present in the CV system that affects CO but the relationship between the amount of blood volume and the Wall Tension (Elastance) of the veins that is controlling Venous Return. Higher amounts of Venous Wall Tension will increase the relative amount of Stressed vs Unstressed Volume in the individual Venous Segments. Relating these venous and arterial vascular parameters to one another is the capability and goal of the present invention.

### Hysteresis in the Data and How it Relates to Stressed and Unstressed Volumes

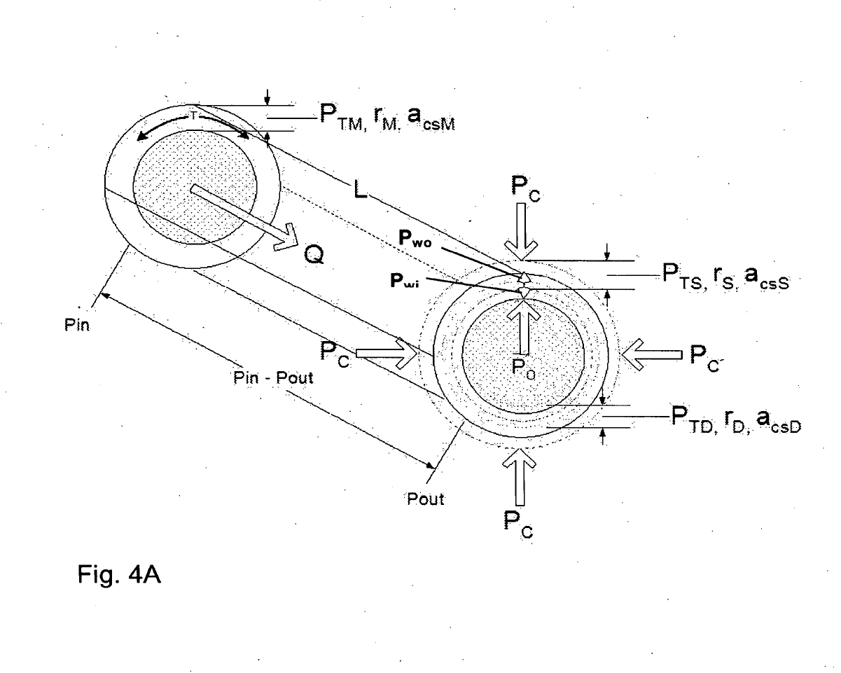

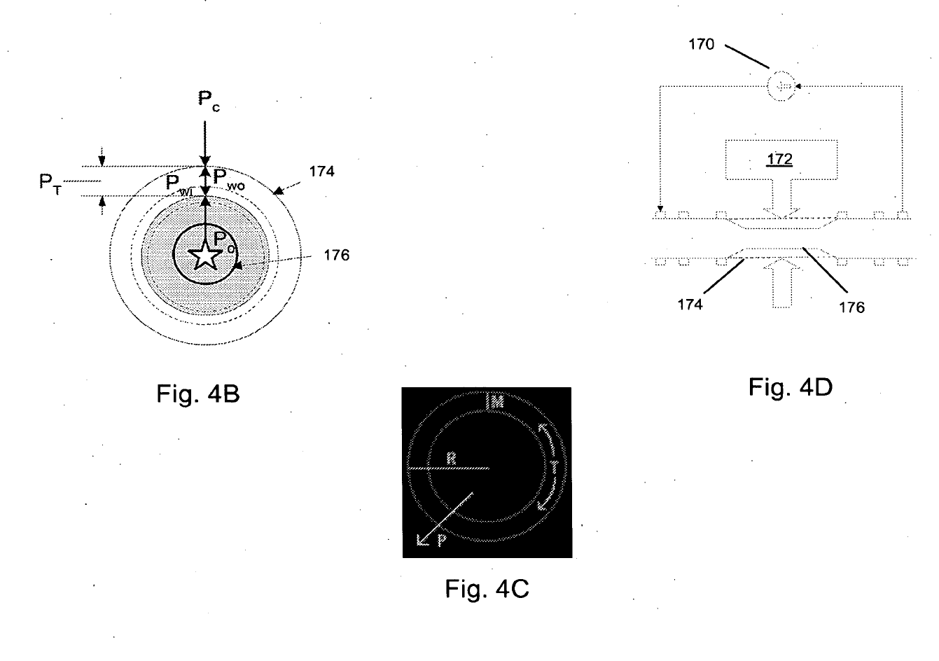

To understand the hysteresis in the Invention data we must relate it to the forces in the wall of the vessel segments as they are depleted and replenished over the course of the cuff pressure cycle. In FIG. 4a, Pwi is the force that is produced by the physiologic control of the muscle fibers in the wall of the vessel. Pwi is believed to be equal to the wall tension ‘T’ described by Laplace's Law (FIG. 4c). Pwi is the primary mechanism of physiologic control used to regulate blood pressure, volume status, and flow through the vessel. Pwo is the mechanical property of the vessel associated with ‘hardness’ or ‘rigidity’ of the wall. It is a function of atherosclerosis and other disease processes commonly thought to occur in blood vessels. Pwo inherently has two components, one is a force vector Pwoo pointing out which represents the forces that resist deformation of the vessel when outside forces such as Pc come against it, the second component is a force vector Pwoi pointing inward which resists the reinflation of the vessel once it has been completely deflated and stretching of the vessel wall once it has reinflated. Pwoi is similar to Pwi in its effect on the vessel behavior but it has a different origin. There is new information to be gained regarding the differences between physical hardening of the diseased blood vessels over time and the physiologic controls that are manipulating the vessel wall tension (T or Pwi) for maintenance of pressure control, volume status, and flow. The clinical opportunity for the Invention is to be able to differentiate between mechanical diseases of the vasculature from diseases affecting physiologic control of the vasculature. The prior being analogous to having rusty pipes and the latter being a physiologic control problem. Both can have adverse affects on the performance of the vasculature but due to differing modes of malfunction. With existing instrumentation methods it might be difficult to tell the difference but not so with the current invention.

T=PT*(R/M)=Pwi;

PT=transmural pressure, R=radius of vessel, M=wall thickness

Pwi/PT=R/M=Pwi/(Po−Pc)

Closing pressure occurs when Pcc>Po+Pwoo

Opening Pressure occurs when Pco<Po−Pwoi

Segment Hysteresis=Pcc−Pco=(Po+Pwoo)−(Po−Pwoi)=Pwoo+Pwoi

We can choose to assume that Pwoo=Pwoi and calculate the average of the two in order to determine the mean pressure associated with the vessel segment, OR we can choose to assume a typical 1/3 vs 2/3 relationship between these two values and use that ratio to calculate the mean pressure value for the vessel segment. The variance between these two calculations would not be significant in any clinical scenario but it would be academically interesting to know the relative values of Pwoo and Pwoi? These two forces in the vessel wall may be significantly involved in the response characteristics of pulsed vessels such as arteries!

### The Conundrum of Pwo, Pwi, Stressed Volume and Unstressed Volume?

The Oscillometric Thesis that was first developed by Dr. Victor Pachon in 1909 France [3] identified the fundamental relationship of maximum compliance of the arteries (maximum pressure pulse amplitude in the cuff) occurring when the outside pressure (Pc) equaled the mean pressure of the vessel. This state as seen in FIG. 4a would make the transmural pressure PT equal to zero mmHg. When we apply this same principle to the veins we see a similar behavior with one exception, there are no ongoing pulsations (pulsatile volume changes) in these vessels which we can use to identify this state as we do in arterial oscillometry. However, the compliance still changes over the range of pressures associated with a vessel segment as does the residual volume levels both of which can be detected by the Invention.

Loring B. Rowell PhD [4] identified two types of residual fluid volume in blood vessels, Stressed and Unstressed! The Unstressed Volume is defined as the amount of volume that fills the vessel segment when the transmural pressure is equal to zero. The Stressed Volume is defined as the amount of volume that fills the vessel segment in addition to the Unstressed Volume in the natural (unmodified) state of the vessel segment! However when we apply these principles to our models in FIG. 4a and FIG. 4b as PT goes to zero, then Pwi must go to zero, or near zero as well. Furthermore, the wall tension T must also minimize in this state as there is no stressed volume remaining in the vessel segment to stress the wall of the vessel.

At this point the only remaining force in our model is Pwo with its duality of Pwoo and Pwoi! Pwo is the rigidity force which is a mechanical resistance to elastic change and/or compression of the vessel! We might view this force as the “Hardness” or “stiffness” of the vessel wall! It appears that this force may be responsible for some or all of the hysteresis between the depletion and the replenishment cycles of the volume/pressure loop response in the Invention data. Furthermore, according to Laplace's Law if Pwi and PT are both zero, Pwo with its duality of Pwoo and Pwoi are the only forces yet in the wall! During depletion the ‘closing pressure’ of the vessel is often higher than the ‘opening pressure’ observed during replenishment.

The relationship between Stressed and Unstressed Volumes in arteries versus veins are an important new indicator of vascular distress or dysfunction. These relationships exemplify how the Invention is a relational instrument rather than just a parameter instrument in aiding clinical diagnosis and treatment!

The hysteresis of the Depletion vs Replenishment volume data may be a direct measurement of the ‘hardness’ of the vessel segments in the PVL. This measurement could be performed as a routine test during an annual medical examination in order to track hardening of the arteries and the veins over the life of an individual. It further could be used by drug companies to observe the effects of medications on these vessel wall parameters in each segment of the PVL.

### Relational Hemodynamics: What Governs the PVL Behavior Relative to Cardiovascular System Performance?

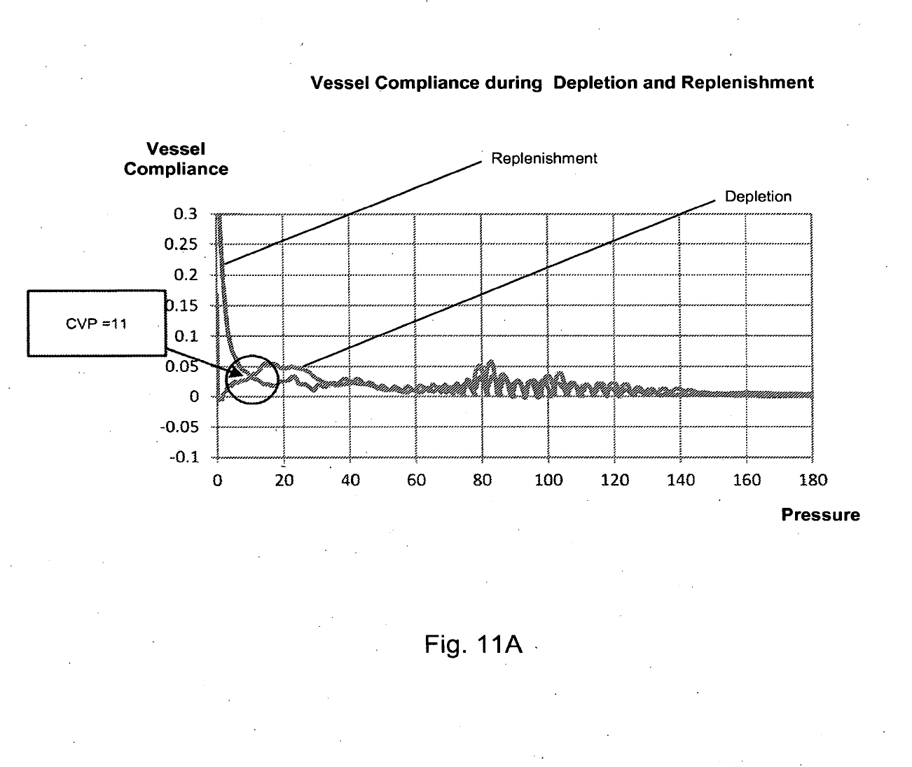

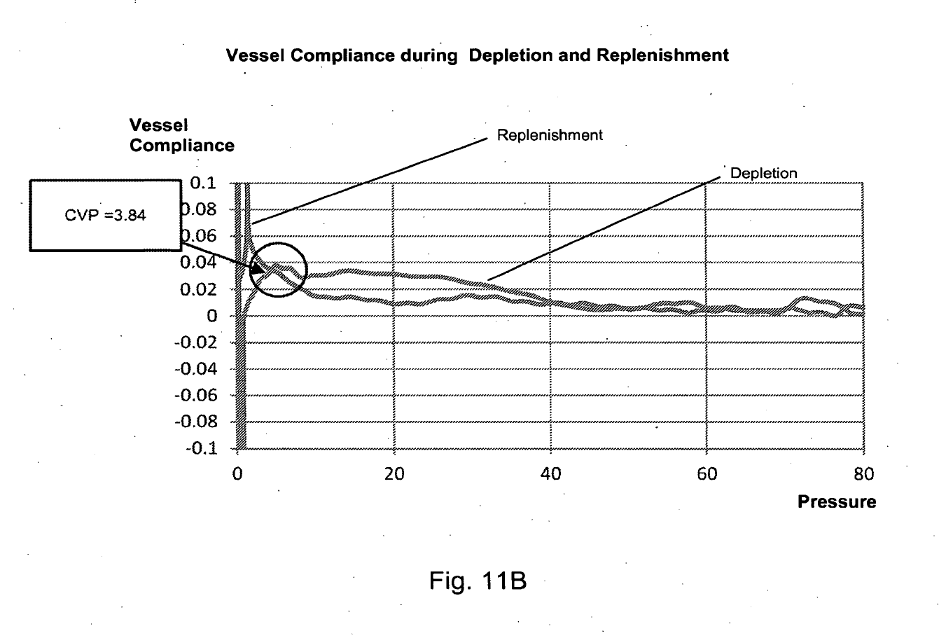

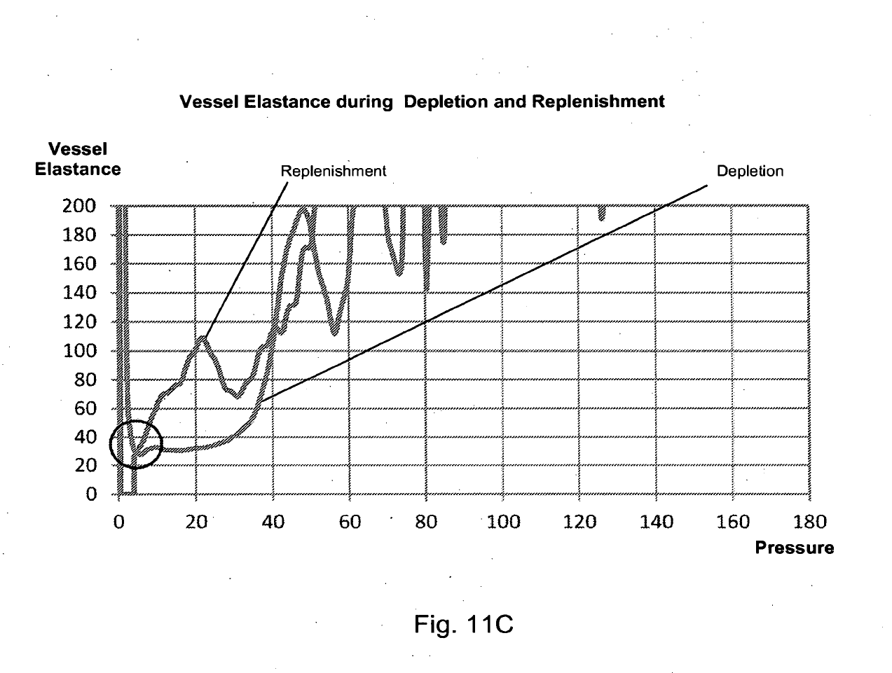

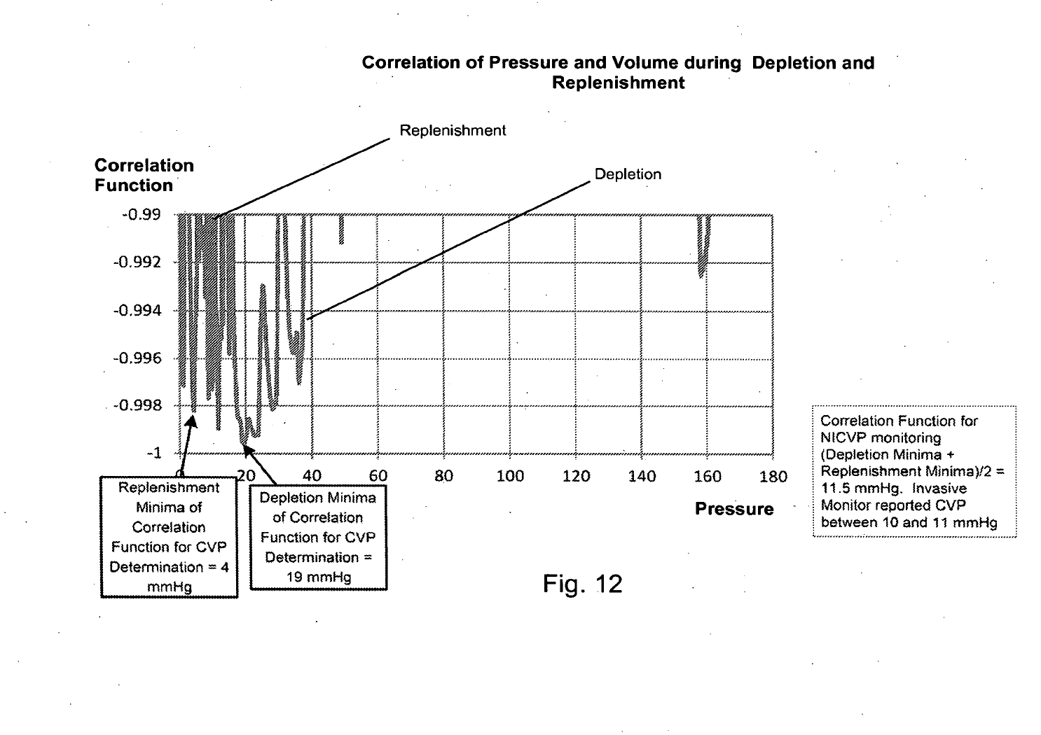

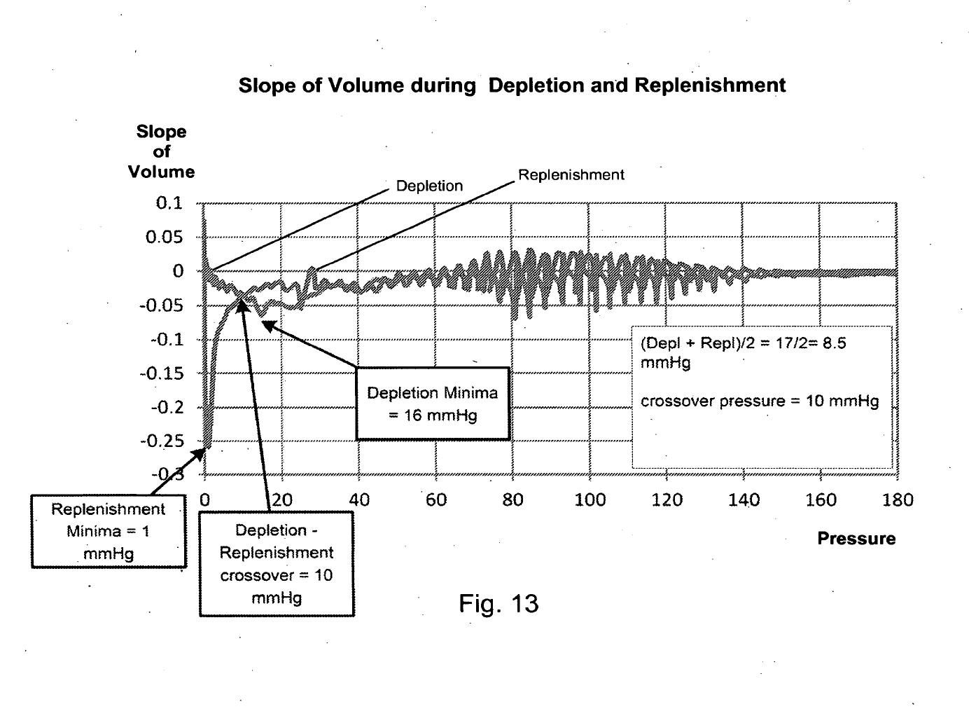

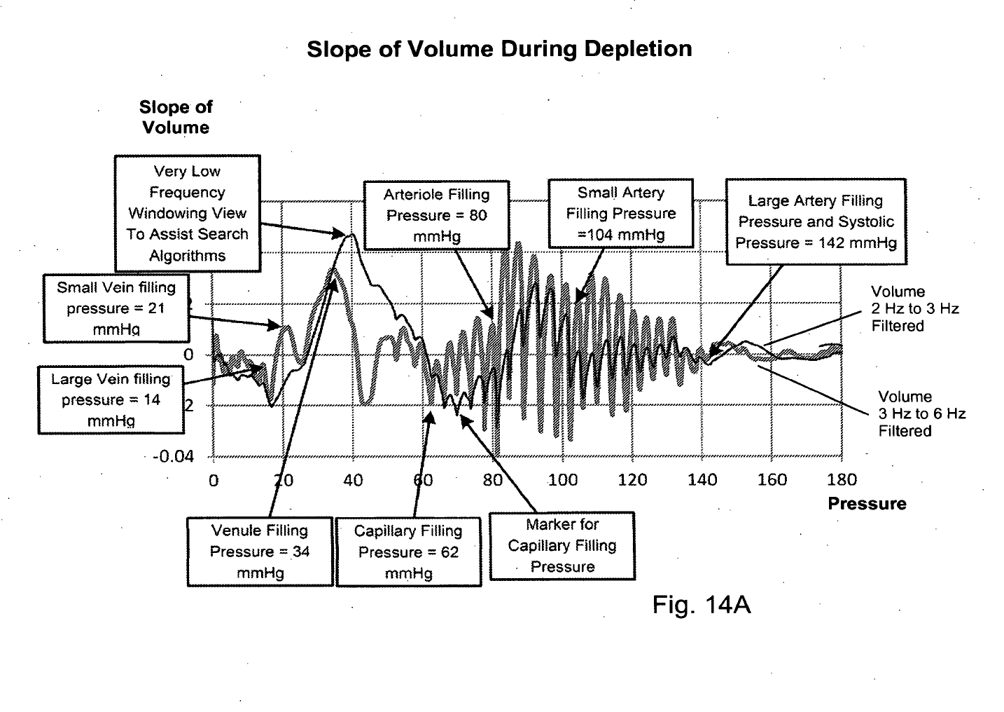

The primary function of the vascular system is to communicate blood from the heart to the capillary beds and back to the heart for recirculation. Therefore, the capillaries can be viewed as the endpoint (objective target) of the cardiovascular system. It is also important to recognize that every cell of the body exists within 2 or 3 cells of a capillary. This means that the network of vessels that communicates blood from the heart to the capillaries is highly divided in order to accomplish this feat. That physical architecture is organized serially because fluid flow by nature occurs serially and the blood vessels involved in that flow must be organized by a means that satisfies the physical laws of nature governing fluid flow. The basic notion of fluid flow is that it flows downhill. The ‘hill’ is the pressure gradient that exists in this network of vessels. Elastance, FIG. 11c, of the vessels is what makes the hill. Therefore, we know that the laws of nature require that there be a pressure drop along the path of flow in FIG. 1. We also know that for arteries, capillaries, and veins to be pressurized they must contain some ‘Residual Volume’ of fluid at all times in order to perform their function. The residual volume resides in the vessel at all times independent of flow through the vessel. It is the residual volume in each vessel type that is establishing its transmural pressure. Flow and Resistance determine the longitudinal pressure gradient in this highly divided network of vessels. Furthermore, the amount of residual volume in each vessel is a function of the hydration status of the patient and the compliance of the vessel wall (i.e. a measure of the capacity of the vessel), the amount of flow volume coming into the vessel and the downstream resistance to flow out of the vessel. It is the residual volume in each stage or segment or compartment of this massive network of vessels that establishes the stratified PVL model shown in FIG. 5 called the Vascular Compliance Stack. These laws of nature affecting the serial organization of volume and pressure in the vascular network apply to all PVL's in the body but not necessarily in an identical organizational structure of the PVL. Each PVL operates according to the demands being placed upon it by the local capillary bed. It seems reasonable to presume that to accomplish adequate perfusion for all tissues in a region of the body that these parallel PVL networks must be behaving by a similar but not necessarily identical paradigm of pressure vs. volume in their management of local flow. The Compliance Inflection Pressures in the Vascular Compliance Stack model identify the pressure values at which vessel compliance behaviors change along the path of flow. It is clear that the compliance state changes occur along the path of flow but it is not yet clear why different people function according to different vascular structures. However, it is clear that the relative organizational structure of the segments of the PVL can both increase and decrease flow in the PVL. It seems reasonable that this new knowledge of the organization of the peripheral vascular bed can lead to new insights into the progression of vascular disease and what it means to be healthy or sick.

### Vascular Elastance and Compliance

Compliance and Elastance are properties of the vessel wall. We can assess these properties on the basis of the resultant residual fluid behaviors in each segment of the vasculature. ΔV is the residual volume within a particular vascular segment and a is the resultant transmural pressure across the vessel wall. Elastance (ΔP/ΔV) is the inverse of Compliance (ΔV/ΔP). Even though Elastance and Compliance, to the casual observer, may seem like two sides of the same coin, however, they affect the performance of the cardiovascular system in different ways because of the physical architecture of the PVL. The blood vessels of the PVL are in constant contentioning between the forces of fluids attempting to move through them and the physiologic controls affecting the character of the vessel walls. Homeostasis is the balance of these competing forces within the body. Increased Elastance of the Veins can increase Preload of the heart which will increase flow (Cardiac Output—CO). Compliance can be viewed like a rubber band stretching, while Elastance can be viewed as a rubber band contracting. Elastance in the arteries adds to the resistance to flow while Elastance in the veins promotes movement of the blood back to the heart for recirculation. Increased Elastance of the Arteries will increase Afterload on the left heart which will decrease flow. Increased Elastance in the veins will increase Venous Return and therefore increase CO. Some vascular controls affect these characteristics of the arteries and veins in opposite directions. For instance, it has been demonstrated that some vasoconstrictor drugs while constricting the arteries, actually dilate the veins. These control elements treat the PVL as if it is like a teeter totter that rotates around a center pressure axle, probably either the Capillary Filling Pressure or the Venule Filling Pressure. Other vascular control elements like nitric oxide (NO) treat the PVL uniformly in all vessel types. Still other elements like caffeine have been shown to affect only the large and small arteries in the limbs without affecting the same vessels in the thorax. These selective behaviors of the vascular controls allow the body to adapt to varying demands and circumstances. However, when those controls become dysfunctional or imbalanced, we have disease.

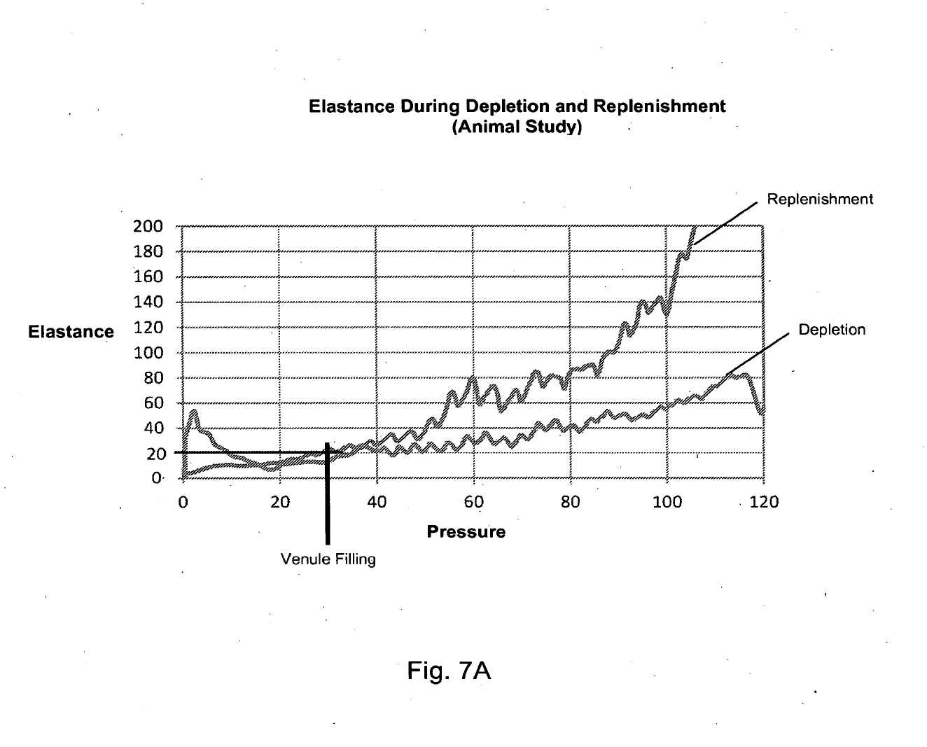

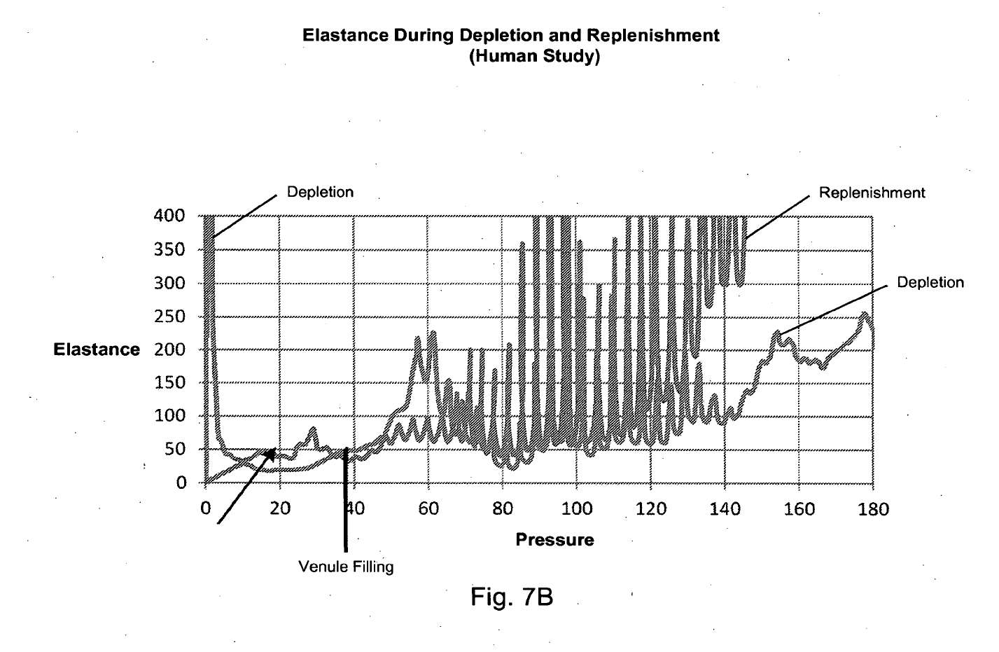

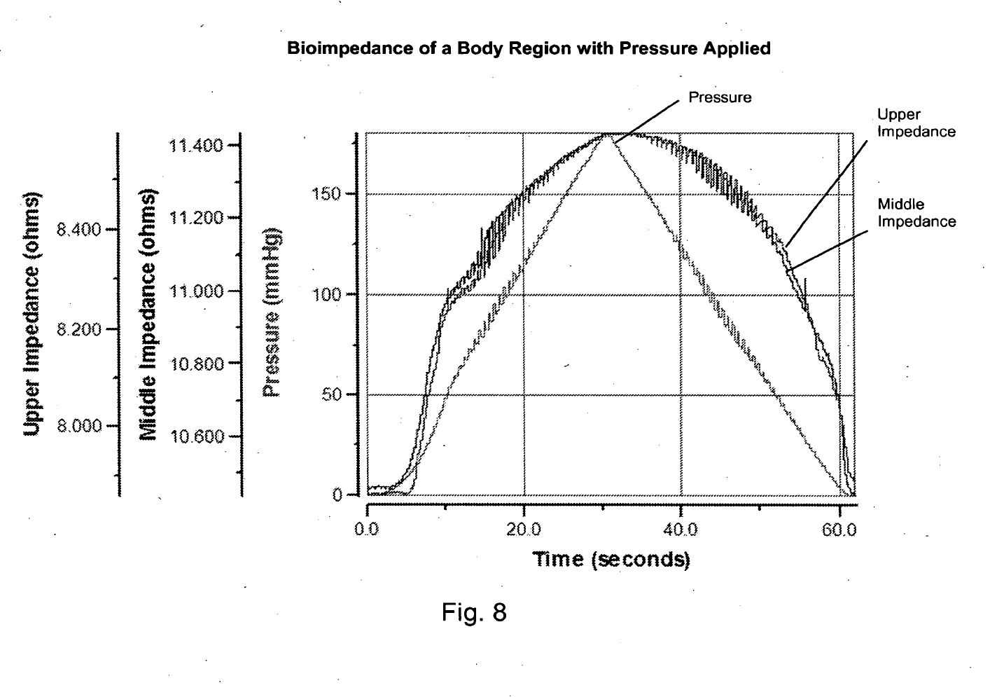

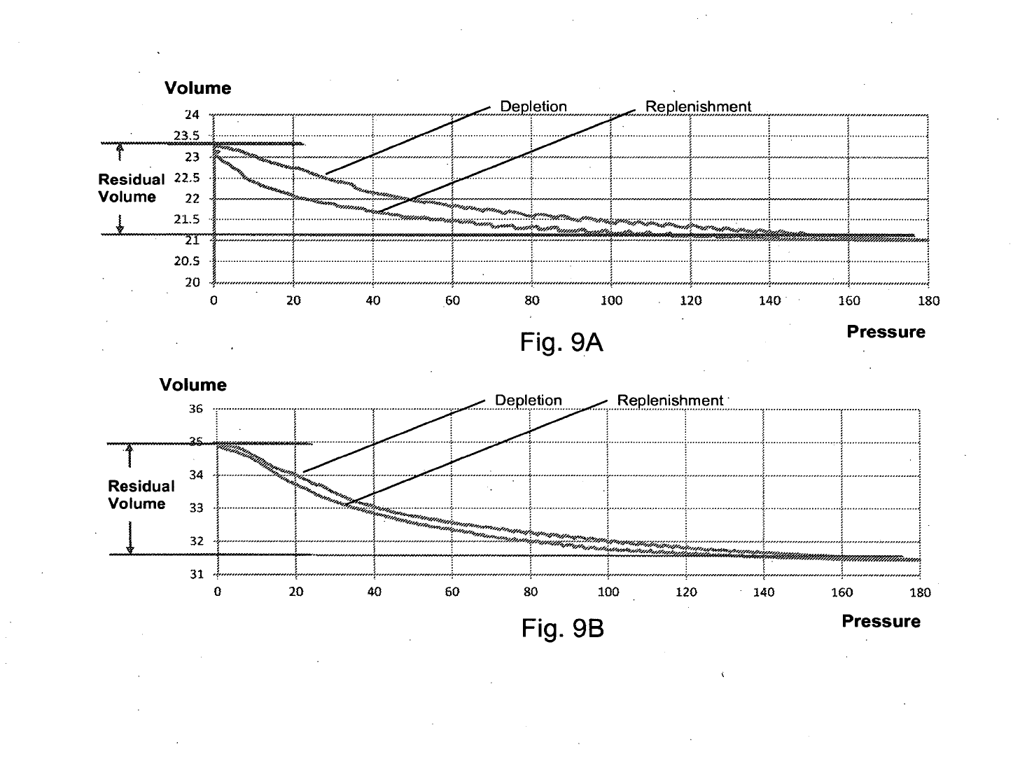

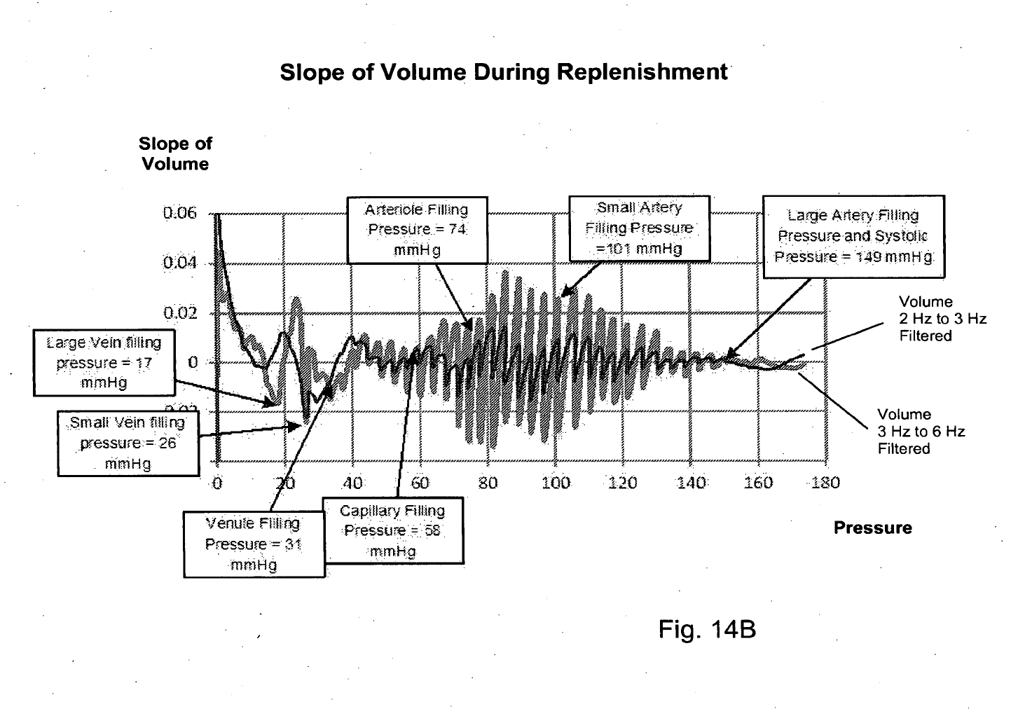

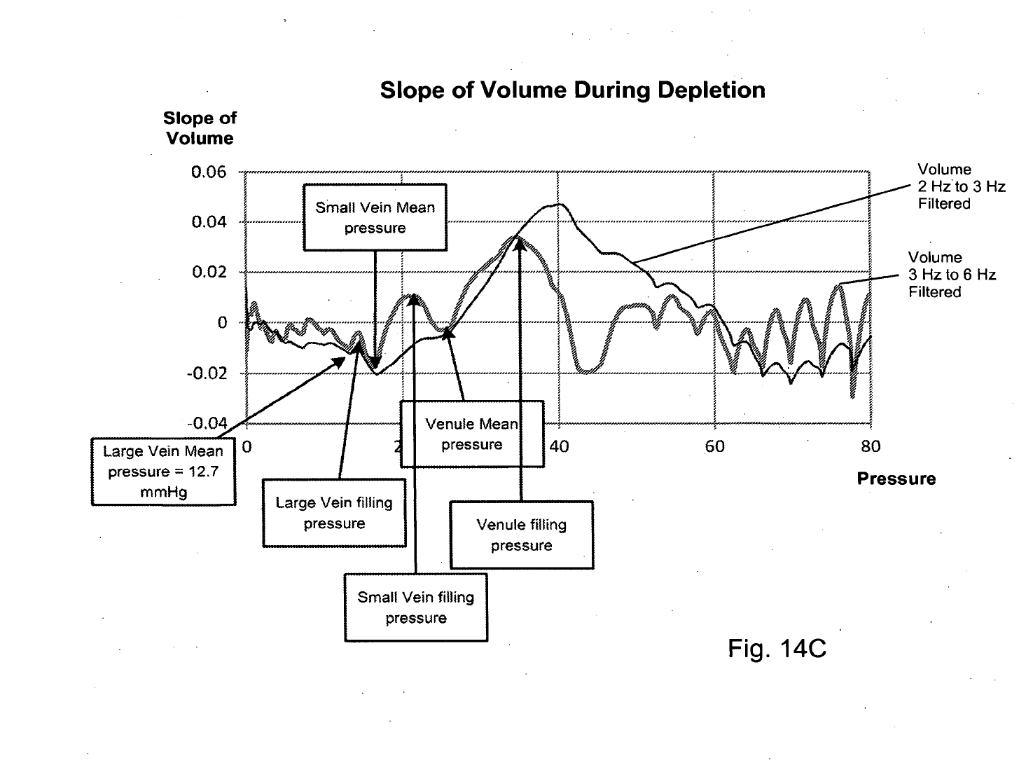

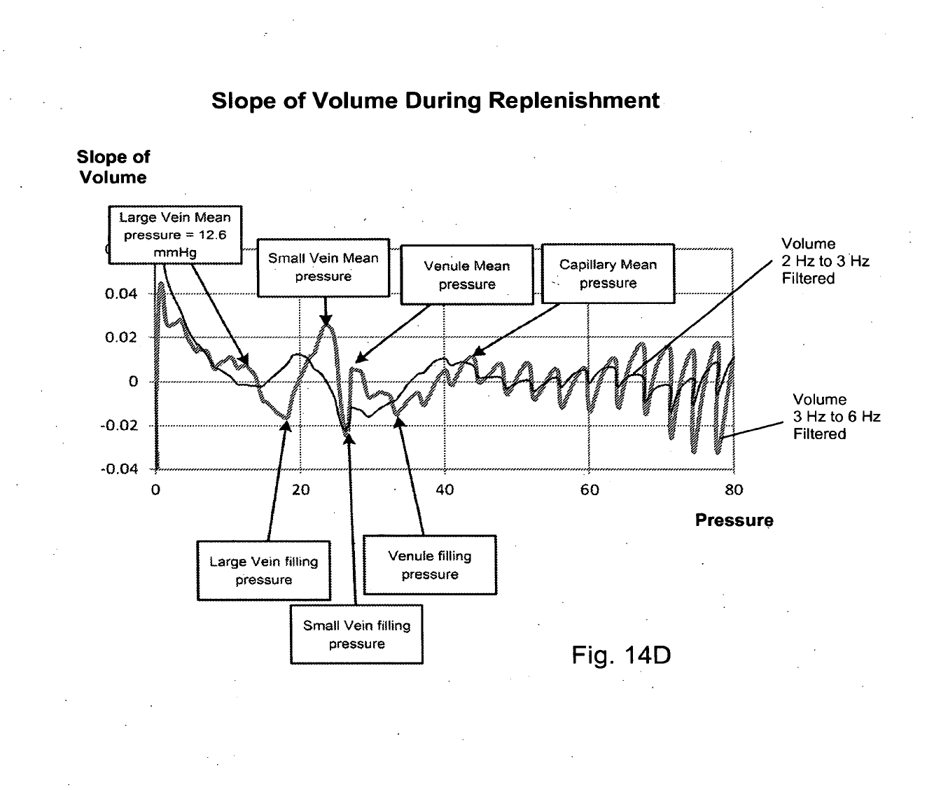

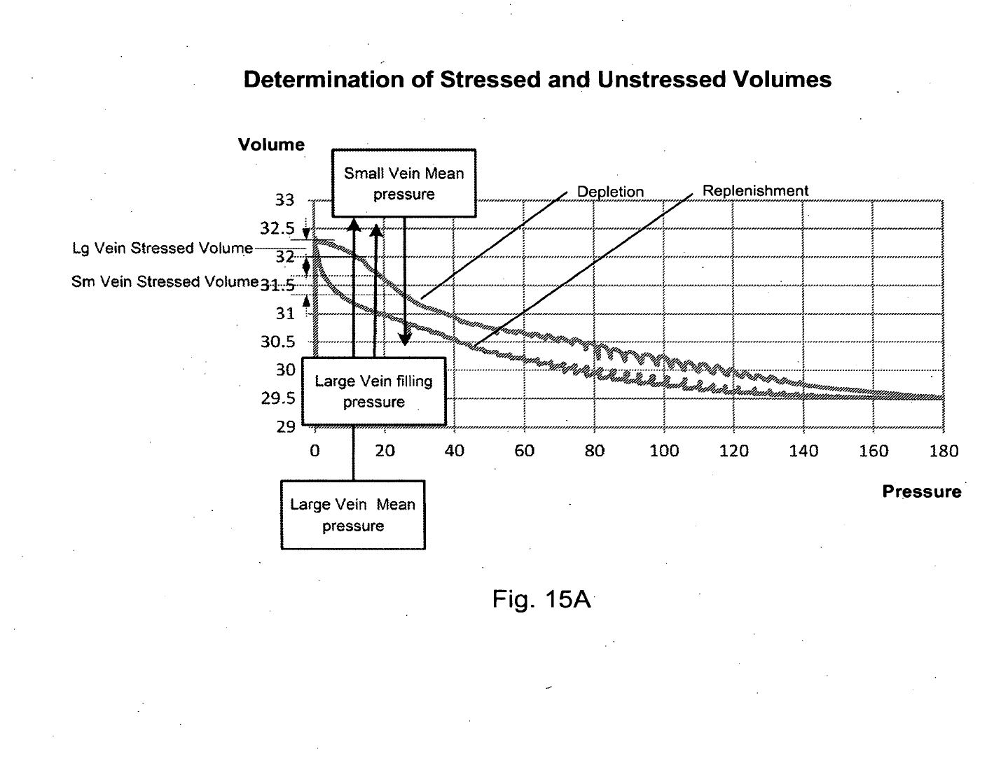

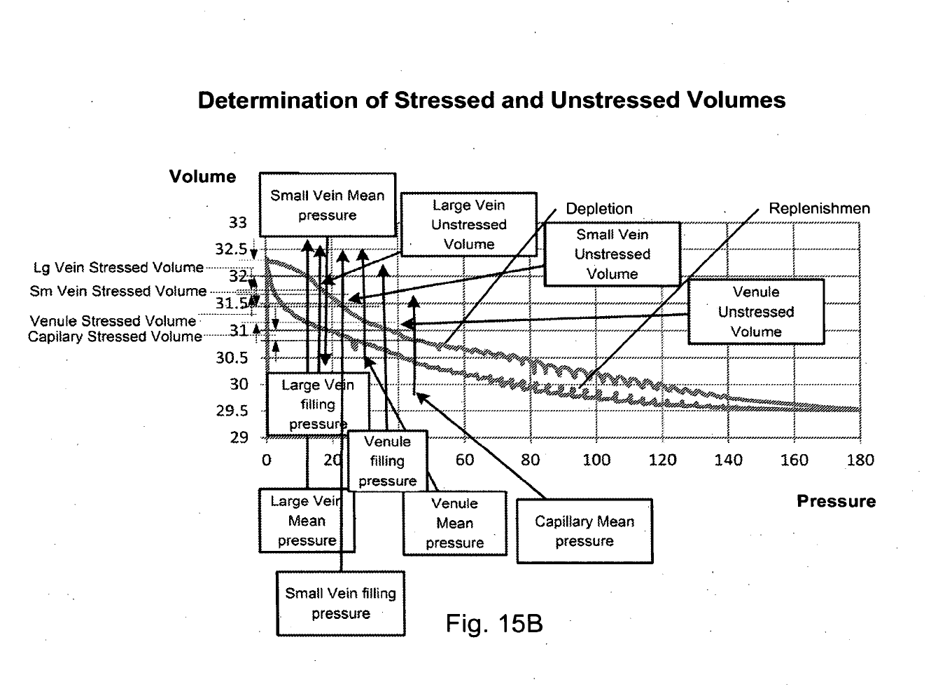

The Sepsis syndrome affects the cardiovascular system by causing systemic dilation of blood vessels, arteries, veins, and capillaries. It is well known that the effects of Sepsis on the cardiovascular system is to first cause an increase in Cardiac Output due to the reduction in venous return prior to the system collapsing due to the reduction in afterload on the heart. However, dilation of the veins (increased Compliance) would have a counter effect on flow as vascular capacity increases and the blood volume sinks into the veins. With reduced Elastance of the veins less blood is returned to the heart and eventually cardiac output diminishes. Due to reduced preload, a downward spiral begins with the arterial blood pressure dropping due to less stroke volume and afterload. The present invention measures the Elastance profile of the PVL (arteries and veins) and therefore can determine the amount of Elastic force presented to the right heart by the venous system. FIG. 9. This Venous Elastance is believed to be the forcing function that produces Preload of the Heart. Preload is currently defined as, ‘the end volumetric pressure that stretches the right or left ventricle of the heart to its greatest geometric dimensions under variable physiologic, demand. ^{[1]}In other words, it is the initial stretching of the cardiomyocytes prior to contraction; therefore, it is related to the sarcomere length at the end of diastole.’ [Wikipedia] Preload is defined as a function of the state of the heart at the end of diastole rather than as the force or mechanism that is producing that state. The Venous Elastance is the attribute of the veins that produces the force behind Venous Return to the Heart as long as the veins have stressed volume. Stressed volume is the volume in the vessel that produces a transmural pressure across the wall of the vessel. The Heart is a nonsucking volume pump which is dependent upon venous return in order to produce cardiac output. Reduced venous return=reduced cardiac output. Being able to measure both the vascular Elastance and also identify the Venule Filling Pressure allows the present invention to be able to determine the Venous Elastance as shown in FIGS. 7a and 7b. The present invention can evaluate the Narrow Band Volume Analysis profile as shown in FIGS. 14a, 14b, 14c, and 14d for the mean pressures of each of the venous segments in order to determine Venous Elastance for compromised Venous Return. The amount of stressed volume in the segment can be determined in the Depletion cycle data by determining the amount of volume that is resident in the segment between first exudation of volume till the segment's mean pressure is detected. On the Replenishment cycle the Stressed Volume is determined between the detection of the mean pressure and the filling pressure of the next segment. Without stressed volume in the veins, venous return would be highly compromised. This capability will be extremely beneficial in the treatment of Shock (especially Sepsis), and Heart Failure. The ratio of stressed vs unstressed volume in a segment would be indicative of the amount of Elastance present in the segment.

This clearly shows the interrelationship between the role of the arteries to produce pressure and the role of the veins to produce return of blood to the heart. The heart may be the pump but if it gets nothing back from the veins, it becomes of little use. The ability of the present invention to measure the structural attributes of both the veins and the arteries relative to one another allows it to identify these critical relationships between the veins and the arteries in order to diagnose disease. A simple ratio of arterial compliance vs venous compliance would be indicative of vascular status and a guide for therapy. The inventor believes that the segmental information (pressure values, compliance values, and elastance values) of the PVL will allow for determination of any number of relative values between segments which are indicative of vascular disease and reduced performance capability.

### Methods of Finding Relevant Information in the Raw Data

It is an object of this invention to segment the PVL data into the functional segments that exist along the pathway of blood flow in the body. The questions of WHAT is controlling various pressures in the PVL will be illustrated by the expressions of the Invention profiles that were filtered at various cutoff frequencies in order to separate the effects of the pulsed activity from the residual activity in the system. The fluid systems of a limb are complex and interactive. In a limb of the body arteries are flowing pulsatile blood in one direction and veins are flowing nonpulsatile blood in the other direction. Respiration effects are present in the raw data. Raw data represents both vascular and non-vascular fluid compartments, all of which must be delineated in our processing methodologies. The vascular system is intimately in communication with nonvascular fluid compartments. Being able to separate signal components that are originating from different sources in the body can be difficult.

It was discovered that most residual volume energy can be observed most effectively in the zero to 0.6 Hz frequency range. The bandpass filtering used in the analysis of the Invention data is often done in the 0.2 to 0.6 Hz range in order to best express the effects of the residual fluids on the structure and function of the vessel segments. A very narrow band of information in the 0.2 to 0.3 Hz hand is useful in grossly identifying the general organizational structure of the PVL so that other algorithms can then find the refined inflection pressures by vessel type. It is very important in the clinical assessment and interpretation of the Invention data to recognize this frequency relationship of the residual vascular volume to the functional structure and behavior of the vascular system. Much can be learned about the vascular controls by studying the residual volumes in this very low (nonpulsatile or residual) frequency band. This band is useful in identifying the primary functional segments of the PVL by identifying the filling pressures of the Venules and the Capillaries.

Most prior noninvasive and invasive medical monitoring technologies are based upon measurements of the pulsatile characteristics of the arteries. These products have no ability to measure the residual fluid volumes in blood vessels and therefore cannot monitor the organization and functionality of the nonpulsed vessels of the PVL, specifically the capillaries and veins. Furthermore, conventional pulsed based technologies cannot assess the residual volumes in the arteries which is often relevant to CV disease (hypertension, migraine headaches, kidney disease, shock, dehydration, excess peripheral resistance, etc.). Due to this lack of information regarding the functionality and performance of the often called “Capacitive Vessels,” very little information about the role of the veins in overall cardiovascular performance is either known or conveyed in medical physiology textbooks.

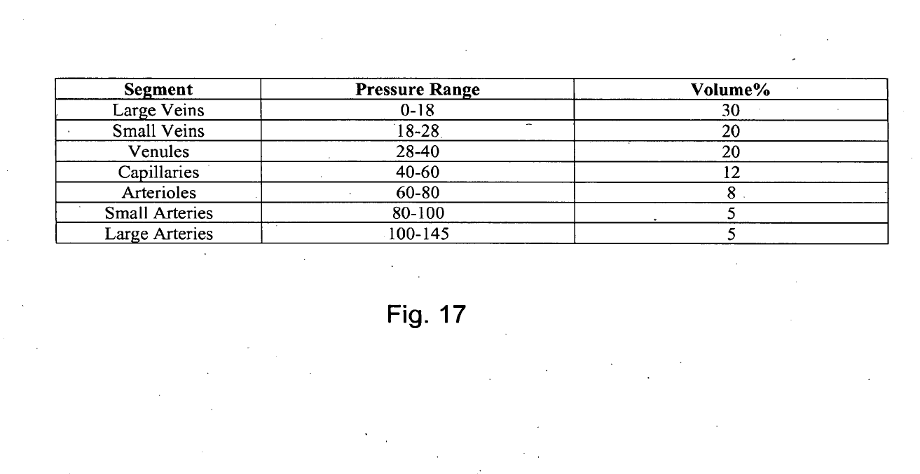

There exist an infinite number of variations on a theme in the profile images produced by the Invention. That is because humans are like snowflakes and fingerprints, all similar with no two being identical! Therefore, the challenge of the Invention is to identify the ‘themes’ (patterns) of the Invention data so that we can make use of that information to properly interpret the parameters of interest. These patterns can be represented by a ‘State Table’ of PVL values which describe the state of the individual PVL. One example of a State Table, FIG. 17, would be the pressure range for each segment in one column with the volume for each segment in the second column for each segment of the PVL. Even though profiles from different people demonstrate different organizational structures, the basic forms of the Volume/Pressure Loop and associated processed profiles have similar forms. FIG. 7. The clinical opportunity resides in the assessment of how those structures affect outcomes, which structures are better than others? When that information is compared to current monitoring parameters it demonstrates just how UNINFORMATIVE and often MISINFORMING current physiologic monitoring methods are in gauging the hemodynamic status of a patient! The underlying premise of the Invention is that it all means something regarding the clinical status of the patient! The challenge is to gain understanding of what that clinical meaning is and how to use it productively in patient care?

The Invention profiles represent the entire PVL structure in the limb of the test subject including veins, capillaries, and arteries. Therefore the clinical interpretation of that structure is much more complex than one has in interpreting a waveform for Peak, Valley, and Mean values as we do in invasive blood pressure monitoring for both arterial and venous pressure determination! The Invention data also adds the dimension of Volume measurement which is far more telling about the physiology of the test subject than is pressure alone. There exists such a wide chasm between the information offered to the clinician by conventional hemodynamic monitoring technologies and the Invention that it forces a new perspective and thought process on the clinical decision tree. The Invention changes how we THINK about the hemodynamic status of the patient! It is much more than just a parameter based instrument, it is a ‘relational instrument’ and tool! It allows clinicians for the first time to see how the arteries and veins are interacting relative to each other in order to produce perfusion to the capillaries!

### A Measurement of Vascular Decompensation and Other Useful Applications of the Correlation of Pressure to Volume in the Peripheral Vascular Loop

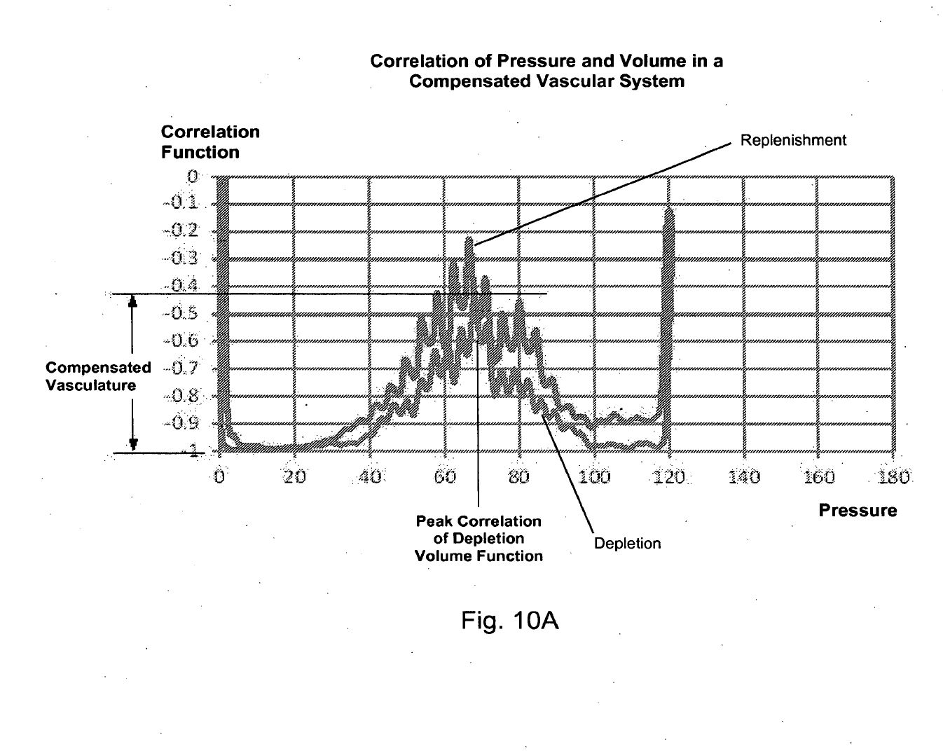

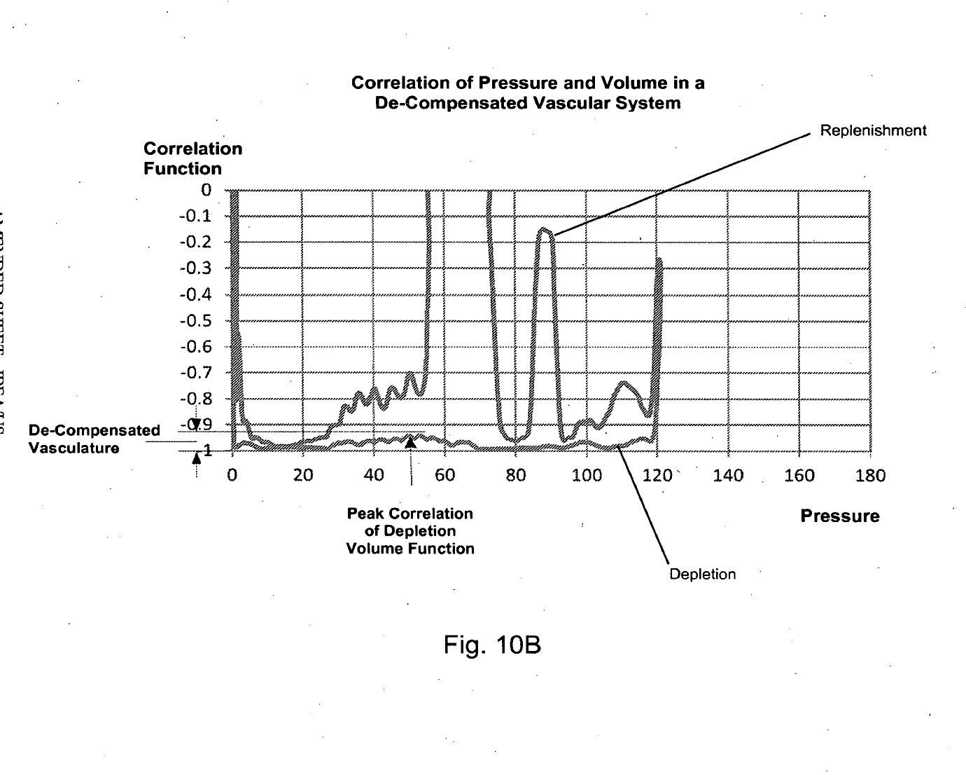

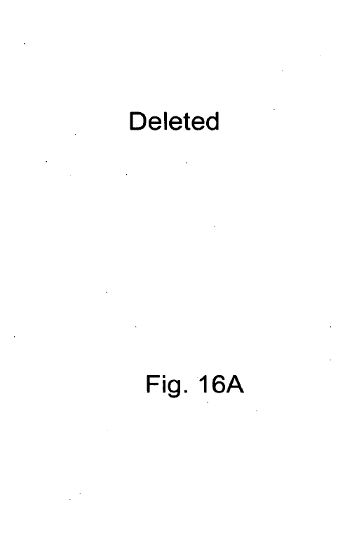

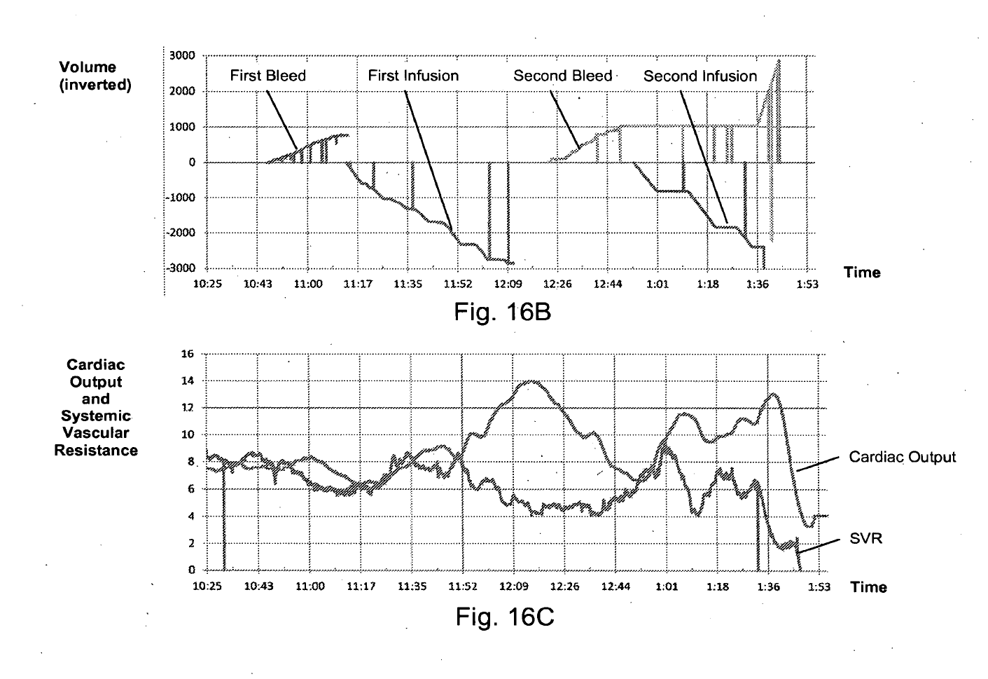

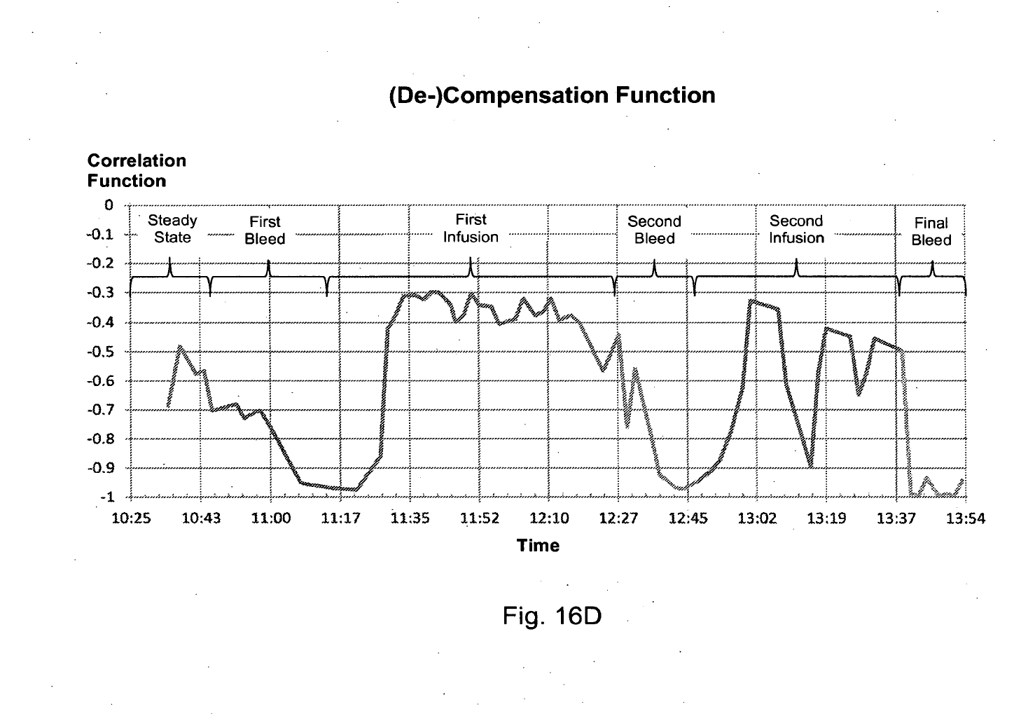

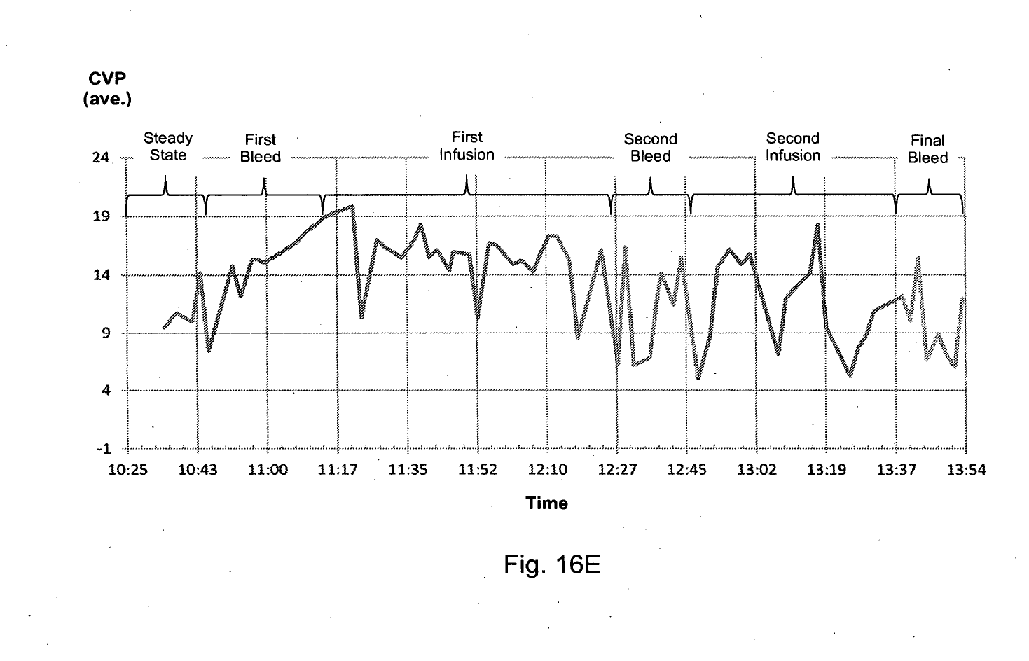

A correlation function compares the similarity of two functions to assess their relationship to one another. In a capacitive medium like blood vessels, the voltage (pressure) lags the current (flow volume) in time. This is called a phase shift. The more phase shift that exists between pressure and volume the more decorrelated the two will be. Correlation functions produce an output value that ranges between 1 and −1 with 1 being very highly similar and going in the same direction and −1 being very highly similar and going in opposite directions and zero being highly dissimilar. In the arteries we have pulsatile data due to the cardiac cycle which exemplifies this phenomenon. The stronger the arterial pulsation, the more de-correlated the pressure and volume signals get relative to one another. This correlation measurement is very useful in determining the strength of contraction of the heart as well as the performance enhancing characteristics of the veins relative to producing venous return. It was determined in a clinical trial (FIGS. 10a and 10b) using a young adult swine that was bled in a controlled fashion until the vasculature decompensated that arterial peak correlation values greater than −0.7 were indicative of a compensated vascular loop and values less than −0.8 were indicative of a decompensated vascular loop. Furthermore, the degree of arterial peak decorrelation from −0.7 to zero was indicative of the strength of flow production from the heart. These values are negative because the pressure lags the volume. The present invention easily determines the correlation value of these two parameters and uses it to assess cardiovascular wellbeing or disease state. The comparison of the Invention data to the Invasive hemodynamic data is presented in FIG. 16b-e. The correlation function, FIG. 16d, between pressure and volume will be extremely valuable in assessing an impending vascular decompensation which frequently occurs in Sepsis and Heart Failure patients thereby allowing medical response prior to vascular collapse. Vascular decompensation can also be determined by the invention by observing the change in vessel compliance or elastance. Vascular decompensation occurs when the blood vessel compliances are much greater than the fluid volume present in the vessels in order to maintain pressure and flow. In other words, there is more vascular capacity than there is blood to fill it up. Homeostasis of the vasculature is achieved when there is a relative balance between the rigidity (Elastance) of the vessel walls and volume of blood in the vessel. Decompensation of the vasculature can occur either when there is rapid blood loss due to hemorrhage, or rapid dilation of the vessels due to chemical imbalances such as found in Sepsis. These conditions can be life threatening to the victim.

### New Methods for Noninvasively Determining Central Venous Pressure (CVP)

Central Venous Pressure (CVP) is the filling pressure of the right heart. It has been used clinically as an indirect measure of Preload [38]. Unfortunately CVP does not always behave the way current vascular models predict and therefore some have challenged its clinical validity [23, 24]. One has to wonder what is the purpose of CVP in the overall control of vascular flow? Here are some things we know about CVP;
1. CVP is the last pressure in the line of vascular pressures produced in the arteries and veins of the body. It is analogous to the caboose of a train (FIG. 2) as it is the pressure left over in the channel of flow, which we call the PVL, when the blood has completed its circuit around the body and returned to the heart. It is measured in or near the right atrium of the heart which by definition means that it is the last pressure value in the PVL.
2. In invasive monitoring, the CVP is generally determined by taking the root mean square value of the invasive pressure wave occurring over the cardiac cycle.

What is not so well known about CVP is;
1. what physically forms CVP in the veins of the PVL,
2. How it relates to the hydration status of the patient,
3. what forces in the vessel control blood pressure, fluid volume status, and flow control
4. how should CVP be interpreted in clinical practice, and
5. what is CVP's purpose in human cardiovascular physiology?

U.S. Pat. No. 6,749,567 disclosed methods for determining CVP on the basis of inflection pressures identified in the slope of volume versus pressure data. Additional methods for noninvasive determination of CVP are disclosed for Narrow Band Low Frequency Volume Analysis, Correlation Function of Volume vs Pressure Analysis, Vascular Elastance Analysis, and Vascular Compliance Analysis. New methods are disclosed for identifying Inflection Pressures in the PVL Volume vs Pressure data.

### Narrow Band Low Frequency Filter Function

A method of Narrow Band Low Frequency Volume Analysis is disclosed which makes use of the frequency relationship of residual volumes to pulse volumes to separate the two for better expression of boundary conditions and Inflection Pressures of the PVL. In FIG. 14 a-d, representative graphs arc shown of the form of this data for both the Depletion and Replenishment phases of the measurement. Key indicators such as maximal, minimas, and zero crossings are used to identify Inflection Pressures for mapping of the PVL and identification of Filling and Mean Pressure values for different segments of the PVL. A Maxima has been identified as the Filling Pressure of the Venules (P_{V}) and a Minima as the Filling Pressure of the Capillaries (P_{C}).

### CVP and Elastance Function

CVP is indicated at the final crossover of the Depletion and Replenishment profiles (Elastance Crossover) of the Elastance function as shown in FIGS. 7b and 11c. These data demonstrate the relationship between Elastance of the veins and the CVP. Furthermore, in controlled animal bleed studies the Elastance Crossover can be observed to increase in Pressure and Elastance as blood volume decreases. This indicator of the Invention is useful in diagnosing and treating cardiovascular disease.

### CVP and Vessel Compliance Function

CVP is indicated at the final crossover of the Depletion and Replenishment profiles (Compliance Crossover) of the Compliance function as shown in FIGS. 11a and 11b. These data demonstrate the relationship between Compliance of the veins and the CVP. Furthermore, in controlled animal bleed studies the Compliance Crossover can be observed to increase in Pressure and decrease in Compliance as blood volume decreases. This indicator of the Invention is useful in diagnosing and treating cardiovascular disease.

### CVP and Correlation Function

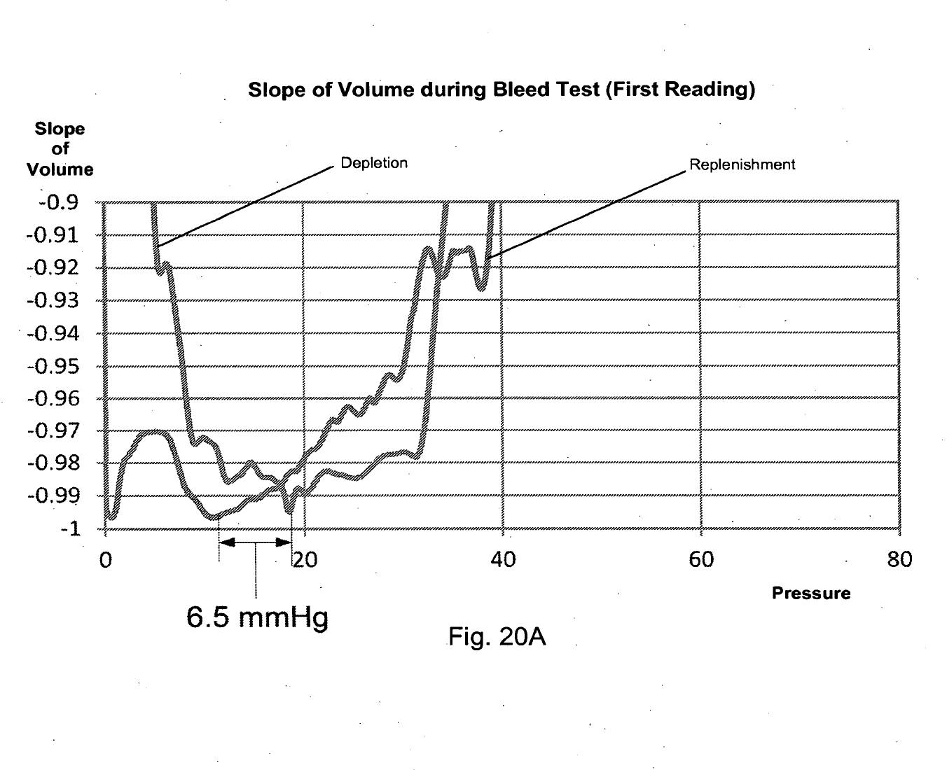

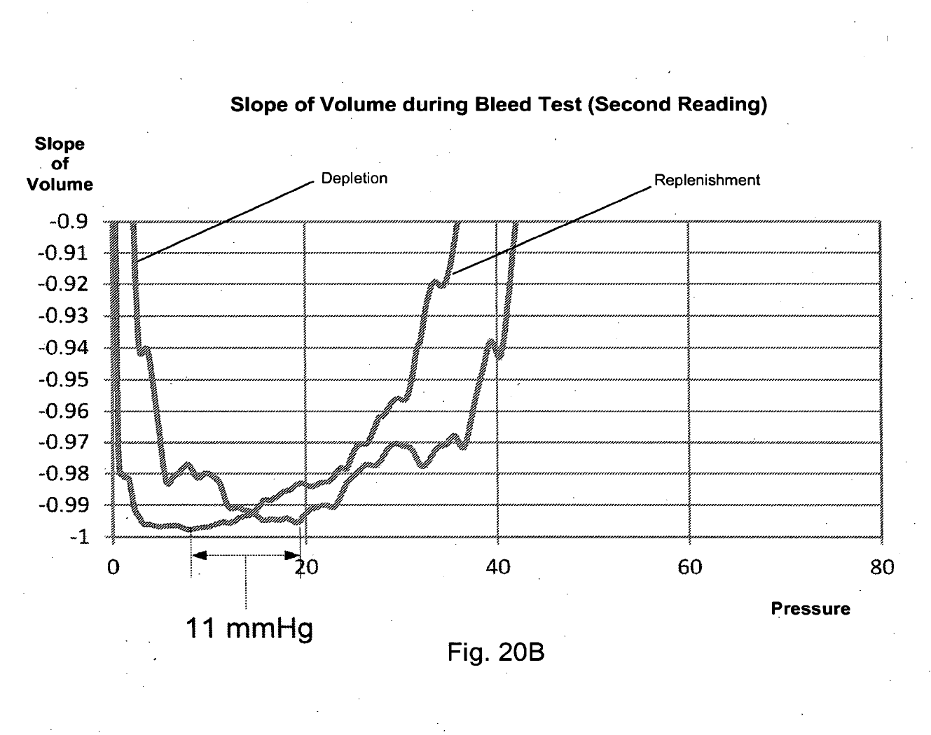

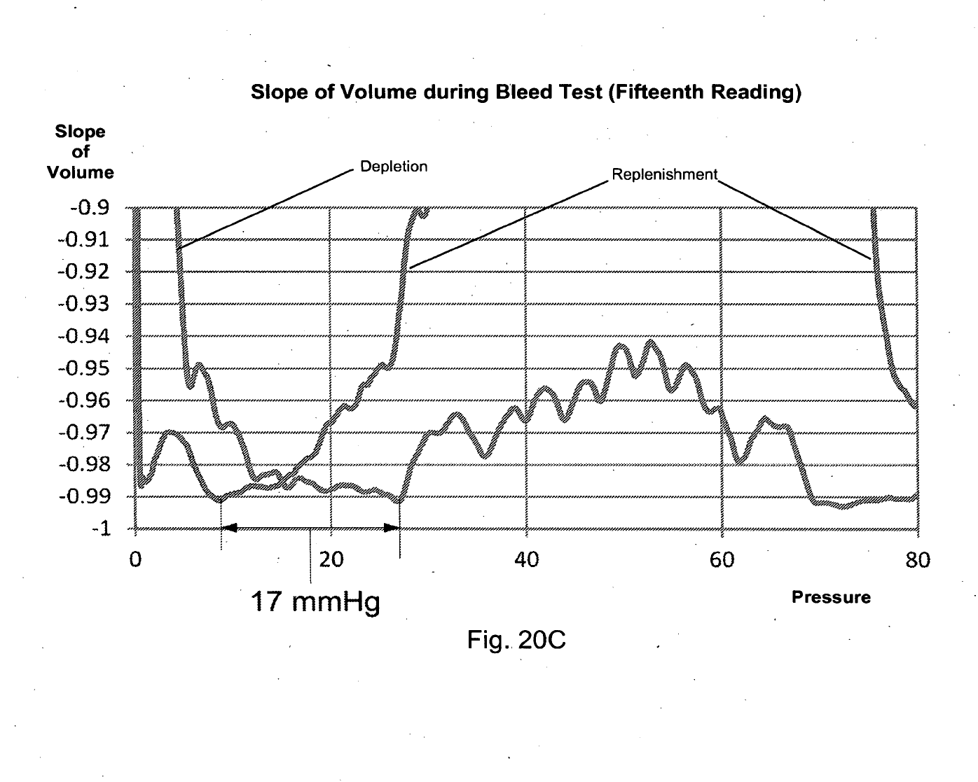

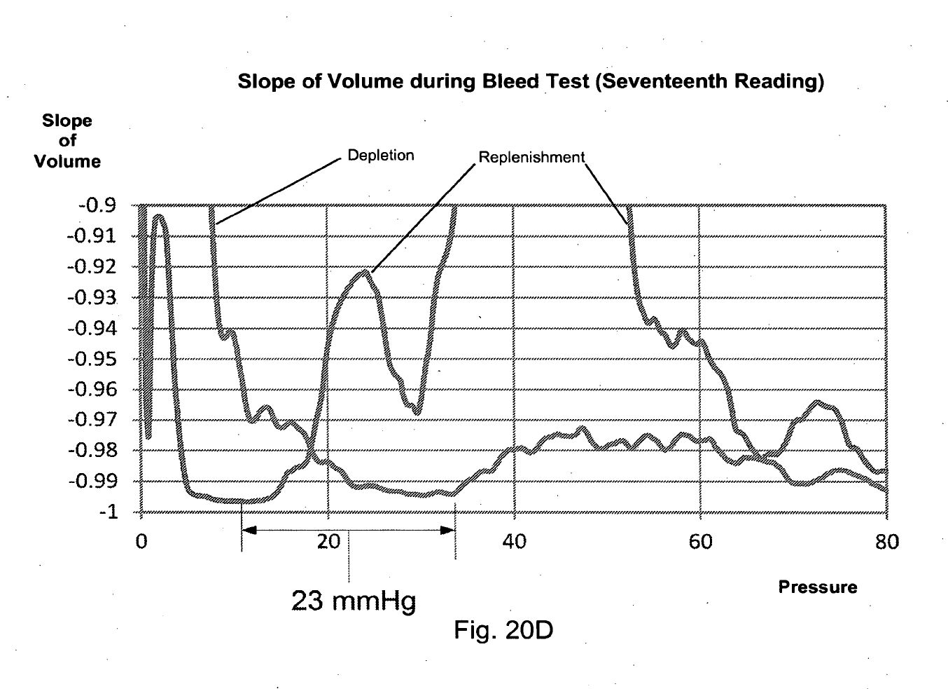

The relationship of the Correlation Function to CVP is indicated at the final crossover of the Depletion and Replenishment profiles (Correlation Crossover) as shown in FIG. 11d. The Correlation Function is different from the Elastance and Compliance Functions relative to CVP determination. The Correlation Function Crossover pressure is more useful in determining the hydration status of the body than it is in determining the CVP per se. These data demonstrate the relationship between the hydration level of the patient and the CVP. The Correlation Crossover Pressures in relation to the Compliance Crossover Pressure or the Elastance Crossover Pressure are an indication of Hydration level in the PVL. In FIG. 20 a-d we demonstrate that in a controlled animal bleed study the Correlation Crossover Pressure is less than the Compliance and Elastance Crossover Pressures when the animal is fully hydrated and one can presume adequate Stressed Volume in the PVL. However, when the animal is bled to a decompensated state in FIGS. 20c and 20d, the Correlation Crossover Pressure is greater than the Compliance and Elastance Crossover Pressures thereby indicating inadequate Stressed Volume in the PVL. Furthermore, the pressure spread between the Depleted Correlation Minima and the Replenishment Correlation Minima gradually increases as the blood is drained out of our test animal. These indicators of the Invention are useful in diagnosing and treating cardiovascular disease and hydration levels.

### Electrical Model of the PVL

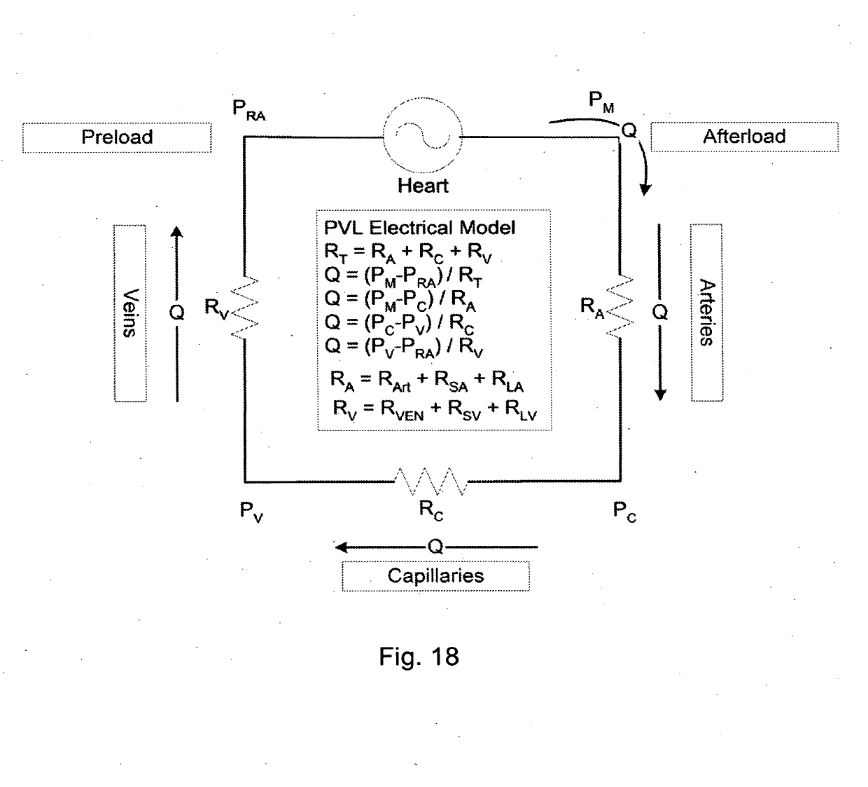

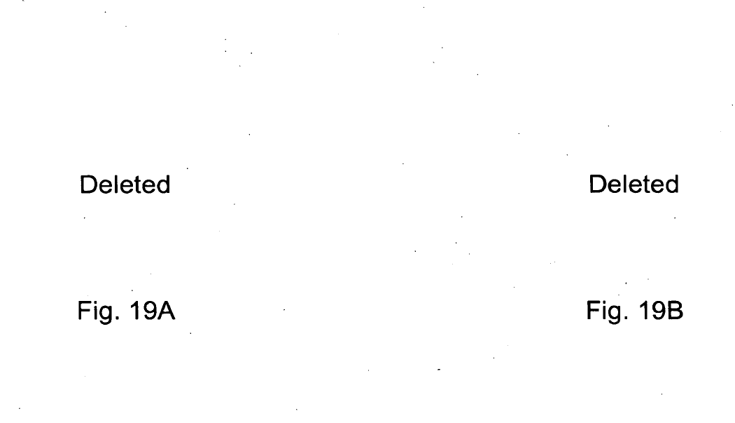

One advantage of the PVL Vascular Model is the ability to view the peripheral vascular organization in a much simplified format as shown in FIG. 18. In this model, the flow (Q), is the same for all segments of the PVL. Lumped resistances for the three functional segments of the PVL, Arteries, Capillaries, and Veins can also be modelled as a single resistance for each functional segment. It can be seen from this model that the resistances of each functional segment are the primary control elements of the PVL in regulating the relative organizational structure of the PVL under any particular demand circumstance presented to the body. This model is important because it illustrates the significance of P_{C} and P_{V} in the values produced by the methods disclosed herein for determining the relative structure of the PVL for any individual body. The methods disclosed herein directly determine Pc and Pv from the Volume vs Pressure data. Pc is the filling pressure of the Capillaries, and Pv is the filling pressure of the Venules or Veins. These are key indicators of overall structure and well being as they are indicative of the relative resistances of the 3 primary functional segments of the PVL. Also from this model we can see that the ratio of resistances in each functional segment of the PVL will affect the overall performance and wellbeing of the cardiovascular system by its effect on overall flow (Q) and the pressures at each of the critical nodes between functional segments. These relationships would be represented in the vascular state table of FIG. 17.

Since we know that the CVP is the last pressure in the PVL and believe that a well-tuned and operating PVL will produce a low CVP, then we know that an elevated CVP means that the flow produced by the heart, Q, and the resistance of the arteries, R_{A}, produce more pressure in the arteries, P_{M}, than is dropped in the total resistance of the PVL across R_{C} and R_{V}. This implies that the combined resistances of the Capillaries and Veins, (R_{C}+R_{V}), are lower than what is necessary by Ohm's Law to drop the pressure at the right atrium to zero. This also implies that the ratio of R_{A}/R_{T} can be an important indication of cardiovascular performance and well-being. The inventor asserts that other ratios (resistance percent of the total) of the relative resistances of the PVL functional segments are indicative of health or disease of the cardiovascular system. Since the filling pressures at the entrance to the Capillaries and Veins are directly measured by the Invention, then relative values of these pressures are also indicative of cardiovascular disease or well-being.

Elevated CVP can also be an indicator that Preload is maximized in the PVL and has exceeded the dynamic range of the Frank-Starling Mechanism of the heart. This indicator of the Invention would be useful in the diagnosis and treatment of Heart Failure. Although Preload is defined in terms of the tension on the Ventricle wall of the Heart at the end of Diastole, it is the force produced by the Veins and the availability of Volume in the Veins that drive Preload to the Heart. This Preload drive can be quantified by the Invention as (P_{V}−P_{RA})/R_{V}. R_{V} is the critical parameter in this relationship since it is the value that is clinically controlled by infused volume and medications. R_{V} is physiologically regulated by endocrine and neurological controls affecting the vessel wall attributes of the veins. Other control loops (associated with the Kidneys) are regulating the fluid volumes in the blood stream.

### FIG. 5

### REFERENCES

1. Guyton A C. Determination of cardiac output by equating venous return curves with cardiac output responses. Physiol Rev 35: 123-129, 1955.
2. Guyton A C, Lindsey A W, and Kaufmann B N. Effect of mean circulatory filling pressure and other peripheral circulatory factors on cardiac output. Am J Physiol 180: 463-468, 1955
3. Douglas Curran-Everett, A classic learning opportunity from Arthur Guyton and colleagues (1955): circuit analysis of venous return Adv. Physiol. Educ. Jun. 1, 2007 31:(2) 129-135
4. Guyton A C, Jones C E, and Coleman T G. Circulatory Physiology: Cardiac Output and Its Regulation. Philadelphia, Pa.: Saunders, 1963
5. Gelman S: Venous function and central venous pressure. Anesthesiology 2008; 108:735-48
6. Young D B, Control of Cardiac Output, San Rafael (Calif.): Morgan & Claypool Life Sciences; 2010.
7. Guyton A C, Abernathy B, Langston J B, Kaufmann B N & Fairchild H M (1959). Relative importance of venous and arterial resistances in controlling venous return and cardiac output. Am J Physiol 196, 1008-1014.
8. Brengelmann G L: Counterpoint: The classical Guyton view that mean systemic pressure, right atrial pressure and venous resistance govern venous return is not correct. J Appl Physiol 2006; 101:1525-6
9. Magder S: Point: The classical Guyton view that mean systemic pressure, right atrial pressure and venous resistance govern venous return is not correct. J Appl Physiol 2006; 101:1523-5
10. Brengelmann G L: A critical analysis of the view that right atrial pressure determines venous return. J Appl Physiol 2003; 84:849-59
11. William R Henderson, Donald E G Griesdale, Keith R Walley, A William Sheel, Clinical review: Guyton—the role of mean circulatory filling pressure and right atrial pressure in controlling cardiac output, Journal: Critical Care—CRIT CARE, vol. 14, no. 6, pp. 1-6, 2010
12. Guyton A C, The relationship of cardiac output and arterial pressure control, Circulation. 1981; 64:1079-1088, doi: 10.1161/01.CIR.64.6.1079
13. C. C. Wo, W. C. Shoemaker, P. L. Appel, M. H. Bishop, H. B. Kram, and E. Hardin, Unreliability of blood pressure and heart rate to evaluate cardiac output in emergency resuscitation and critical illness,” Critical Care Medicine, vol. 21, no. 2, pp. 218-223, 1993.
14. Dellinger, R P; Levy, M M; Carlet, J M; Bion, J; Parker, M M; Jaeschke, R; Reinhart, K; Angus, D C; Brun-Buisson, C; Beale, R; Calandra, T; Dhainaut, J F; Gerlach, H; Harvey, M; Marini, J J; Marshall, J; Ranieri, M; Ramsay, G; Sevransky, J; Thompson, B T; Townsend, S; Vender, J S; Zimmerman, J L; Vincent, J L (2008 January). “Surviving Sepsis Campaign: international guidelines for management of severe sepsis and septic shock: 2008.”. Intensive care medicine 34 (1): 17-60. PMID 18058085.
15. Abraham, Edward; Singer, Mervyn (2007). “Mechanisms of sepsis-induced organ dysfunction”. Critical Care Medicine 35 (10): 2408-16. doi:10.1097/01.CCM.0000282072.56245.91. PMID 17948334
16. Vincent J L, Abraham E: The last 100 years of sepsis. Am J Respir Crit Care Med 2006; 173:256-263
17. Wheeler A P, Bernard G R, Thompson B T, et al. Pulmonary-artery versus central venous catheter to guide treatment of acute lung injury. N Engl J Med. 2006; 354:2213-24.
18. Janssens U, Graf J., Volumenstatus und zentraler Venendruck [Volume status and central venous pressure], Der Anaesthesist May 2009, Volume 58, Issue 5, pp 513-519
19. Jayant, Aveek, What Is Simple Is Perhaps Not Always the Truth, Anesthesiology: November 2008—Volume 109—Issue 5—pp 933-934 doi: 10.1097/ALN.0b013e3181895dd0
20. Schrier R W, Abraham W T. Hormones and hemodynamics in heart failure. N Engl J Med. 1999 August 19; 341(8):577-85.
21. Jacobsohn E, Chorn R, O'Connor M, The role of the vasculature in regulating venous return and cardiac output: historical and graphical approach, CAN J ANAESTH 1997/44:8/pp 849-867
22. Bressack M A, Raffin T A, Importance of Venous Return, Venous Resistance, and Mean Circulatory Pressure in the Physiology and Management of Shock, CHEST/92/5/November, 1987
23. Marik P E; Baram M; and Vahid B, Does CVP Predict Fluid Responsiveness?, CHEST/134/1/July, 2008
24. Marik P E, Surviving sepsis: going beyond the guidelines, Annals of Intensive Care 2011, 1:17 http://www.annalsofintensivecare.com/content/1/1/17
25. Napoli A M, Physiologic and Clinical Principles behind Noninvasive Resuscitation Techniques and Cardiac Output Monitoring,
26. E. Rivers, B. Nguyen, S. Havstad et al., “Early goal-directed therapy in the treatment of severe sepsis and septic shock,” The New England Journal of Medicine, vol. 345, no. 19, pp. 1368-1377, 2001.
27. Fung, Y. C, Biomechanics: Circulation 2nd Edition ISBN 10 0387943846 ISBN 13 9780387943848.
28. Rothe C F, Point-Counterpoint: Active venoconstriction is/is not important in maintaining or raising, end-diastolic volume and stroke volume during exercise and orthostasis J. Appl. Physiol. Oct. 1, 2006 101:(4) 1262-1264
29. Gelman S, Venous Function and Central Venous Pressure A Physiologic Story, Anesthesiology 2008; 108:735-48.
30. Rothe C F and Gaddis M L. Autoregulation of cardiac output by passive elastic characteristics of the vascular capacitance system. Circulation 81:360-368, 1990.
31. Hainsworth R. The importance of vascular capacitance in cardiovascular control. News Physiol Sci 5: 250-254, 1990.
32. Rowell L B. Human Cardiovascular Control. New York: Oxford University Press, 1993
33. Rowell L B. Human Circulation: Regulation during Physical Stress. The Venous System. New York: Oxford Univ. Press, chapt. 3, 1986
34. Reddi B A and Carpenter R H. Venous excess: a new approach to cardiovascular control and its teaching. J Appl Physiol 98: 356-364, 2005.
35. Sheriff D D and Mendoza J R. Passive regulation of cardiac output during exercise by the elastic characteristics of the peripheral circulation. Exerc Sport Sci Rev 32: 31-35, 2004
36. Balmain S, Padmanabhan N, Ferrell W R, Morton J G, McMurray J J V: Differences in arterial compliance, microvascular function and venous capacitance between patients with heart failure and either preserved or reduced left ventricular systolic function. Eur J Heart Fail 2007; 9:865-71
37. Rothe C F and Gersting J M, CARDIOVASCULAR INTERACTIONS: AN INTERACTIVE TUTORIAL AND MATHEMATICAL MODEL, ADV PHYSIOL EDUC 26: 98-109;2002; 10.1152/advan.00031.2001
38. Norton J M, TOWARD CONSISTENT DEFINITIONS FOR PRELOAD AND AFTERLOAD—, Advan in Physiol Edu 25:53-61, 2001
39. Guyton and Hall, Textbook of Medical Physiology 10^{th} Ed., WB Saunders Co., Philadelphia, 2000
40. Jos R. C. Jansen, Jacinta J. Maas and Michael R. Pinsky, Bedside assessment of mean systemic filling pressure, Current Opinion in Critical Care 2010, 16:231-236
41. H. Shimazu, K. Yamakoshi, T. Togawa, M. Fukuoka, “Evaluation of Parallel Conductor Theory for Measuring Human Limb Blood Flow by Electrical Admittance Plethysmography”, January 1981
42. Julian Wright, Alastair Hutchison, Manchester Institute of Nephrology and Transplantation, Manchester Royal Infirmary, Manchester, UK, “Cardiovascular disease in patients with chronic kidney disease,” Vascular Health and Risk Management 2009:5 713-722, ©2009 Wright and Hutchison, publisher and licensee Dove Medical Press Ltd
43. Klabunde, R E, http://www.cvphysiology.com/Blood%20Pressure/BP004.htm

### KEY TO DRAWING REFERENCE NUMBERS

PVL 100

Left Heart Ventricle 102

Large Artery 104

Small Artery 106

Arteriole 108

Capillary 110

Venule 112

Small Vein 114

Large Vein 116

Right Heart Ventricle 118

Direction of Blood Flow 120

Pulsatile Data 122

non-pulsate capacitive vessel 124

Blind zone 126

Interstitium 128

Arterial Blood Pressure 130

Small Artery Blood Pressure 132

Arteriole Blood Pressure 134

Capillary Blood Pressure 136

Venule Blood Pressure 138

Small Vein Blood Pressure 140

Central Venous Pressure CVP 142

Compliance Inflection Pressure 144

Resistance Against Flow 146

Resistance With Flow 148

Volume/Flow Regulator 150

Pressure Regulator/Flow

Motivator 151

Vein Wall Tension 152

Artery Wall Tension 154

Various PVLs of the Body 156

Body region of limb 158

Right Arm 160

Splanchnic Bed 162

Left Arm 164

Aorta 166

Vena Cava 168

AC Constant Current Source 170

Cuff Pressure 172

Inflated Vessel 174

Deflated Vessel 176

Arterial Blood Pressure 178

What is claimed is:

1. A method of obtaining a physiological parameter of a vascular system of a patient, comprising the steps of:
a. applying a series of pressure values to a body region containing a body fluid to deplete and replenish a fluid volume from the body region;
b. sensing the body region under pressure to derive a series of body fluid volume indication values for a plurality of body fluid compartments in the body region under pressure;
c. referencing the body fluid volume indication values of the plurality of body fluid compartments to the series of pressure values, associating each of the body fluid volume indication values to one of the pressure values; and
d. identifying a relationship of the body fluid volume indication values referenced to the series of pressure values, thereby indicating the physiological parameter of the vascular system of the patient.

2. The method of claim 1 wherein the step of identifying the relationship of the body fluid volume indication values referenced to the series of pressure values, thereby indicating the physiological parameter of the vascular system of the patient further comprises the steps of:
e. generating a series of elastance values, each elastance value associated with one of the pressure values, wherein each elastance value is a change in pressure at the associated pressure value divided by a change in body fluid volume indication value at the associated pressure value;
f. generating a depletion elastance graph by graphing the series of elastance values versus the series of pressure values during depletion; and
g. generating a replenishment elastance graph by graphing the series of elastance values versus the series of pressure values during replenishment.

3. The method of claim 2 wherein the step of identifying the relationship of the body fluid volume indication values referenced to the series of pressure values, thereby indicating the physiological parameter of the vascular system of the patient further comprises the step of:
h. identifying a lowest crossover pressure value as central venous pressure, wherein the lowest crossover pressure value is a lowest of the series of pressure values at which the depletion elastance graph and the replenishment elastance graph crossover one another.

4. The method of claim 1 wherein the step of identifying the relationship of the body fluid volume indication values referenced to the series of pressure values, thereby indicating the physiological parameter of the vascular system of the patient further comprises the steps of:
i. generating a series of compliance values, each compliance value associated with one of the pressure values, wherein each compliance value is a change in body fluid volume indication value at the associated pressure value divided by a change in pressure at the associated pressure value;
j. generating a depletion compliance graph by graphing the series of compliance values versus the series of pressure values during depletion; and
k. generating a replenishment compliance graph by graphing the series of compliance values versus the series of pressure values during replenishment.

5. The method of claim 4 wherein the step of identifying the relationship of the body fluid volume indication values referenced to the series of pressure values, thereby indicating the physiological parameter of the vascular system of the patient further comprises the steps of:
l. identifying a lowest crossover pressure value as central venous pressure, wherein the lowest crossover pressure value is a lowest of the series of pressure values at which the depletion compliance graph and the replenishment compliance graph crossover one another.

6. The method of claim 1 wherein sensing the body region under pressure to derive a series of body fluid volume indication values further comprises:
m. filtering raw body fluid volume data to filter out signal components with frequencies above a first threshold and below a second threshold.

7. The method of claim 1 wherein sensing the body region under pressure to derive a series of body fluid volume indication values further comprises:
n. filtering the series of body fluid volume indication values to filter out signal with frequencies above a 0.3 Hz and below 0.2 Hz.

8. The method of claim 1 wherein sensing the body region under pressure to derive a series of body fluid volume indication further comprises:
o. filtering the series of body fluid volume indication values to filter out signal with frequencies above 0.6 Hz and below 0.2 Hz.

9. The method of claim 1 wherein sensing the body region under pressure to derive a series of body fluid volume indication values further comprises:
p. filtering the series of body fluid volume indication values and pressure values to filter out signal with frequencies above a first threshold and below a second threshold.

10. The method of claim 1 wherein sensing the body region under pressure to derive a series of body fluid volume indication values further comprises:
q. filtering the series of body fluid volume indication values and pressure values to filter out signal with frequencies above 0.3 Hz and below 0.2 Hz.

11. The method of claim 1 wherein sensing the body region under pressure to derive a series of body fluid volume indication values further comprises:
r. filtering the series of body fluid volume indication values and pressure values to filter out signal with frequencies above 0.6 Hz and below 0.2 Hz.

12. The method of claim 1 wherein the step of identifying the relationship of the body fluid volume indication values referenced to the series of pressure values, thereby indicating the physiological parameter of the vascular system of the patient further comprises the steps of:
s. generating a series of correlation values, each correlation value associated with one of the pressure values, wherein each correlation value is an output of a correlation function with the body fluid volume indication value as a first input of the correlation function and the associated pressure value as a second input of the correlation function;
t. defining a depletion correlation graph as comprising a first subset of the series of correlation values and associated pressure values, the first subset of the series of correlation values based on body fluid volume indication values obtained during depletion;
u. determining a peak depletion correlation value, wherein the peak depletion correlation value is the correlation value at a maxima of the depletion correlation graph; and
v. determining that the vascular system of the patient is compensated or decompensated based on the peak depletion correlation value.

13. The method of claim 12 wherein the step of determining that the vascular system of the patient is compensated or decompensated based on the peak depletion correlation value further comprises the steps of:
w. determining that the vascular system of the patient is compensated if the peak depletion correlation value is above a first depletion correlation threshold: and
x. determining that the vascular system of the patient is decompensated if the peak depletion correlation value is at or below a second depletion correlation threshold.

14. The method of claim 13 wherein,
y. the first depletion correlation threshold is −0.7 and the second depletion correlation threshold is −0.8.

15. The method of claim 1 wherein the step of identifying the relationship of the body fluid volume indication values referenced to the series of pressure values, thereby indicating the physiological parameter of the vascular system of the patient further comprises the steps of:
z. generating a series of correlation values, each correlation value associated with one of the pressure values, wherein each correlation value is an output of a correlation function with the body fluid volume indication value as a first input of the correlation function and the associated pressure value as a second input of the correlation function;
aa. defining a depletion correlation graph as comprising a first subset of the series of correlation values and associated pressure values, the first subset of the series of correlation values based on body fluid volume indication values obtained during depletion;
ba. defining a replenishment correlation graph as comprising a second subset of the series of correlation values and associated pressure values, the second subset of the series of correlation values based on body fluid volume indication values obtained during replenishment;
ca. determining a lowest correlation crossover pressure value, wherein the lowest correlation crossover pressure value is a lowest of the series of pressure values at which the depletion correlation graph and the replenishment correlation graph cross over one another;
da. determining a lowest compliance crossover pressure value; and
ea. determining a hydration status of the patient based on the lowest correlation crossover pressure value and the lowest compliance crossover pressure value.

16. The method of claim 15 wherein the step of determining a hydration status of the patient based on the lowest crossover correlation value and the lowest compliance crossover pressure value further comprises the step of:
fa. determining the patient is adequately hydrated if the lowest correlation crossover pressure value is less than the lowest compliance crossover pressure value.

17. The method of claim 1 wherein the step of identifying the relationship of the body fluid volume indication values referenced to the series of pressure values, thereby indicating the physiological parameter of the vascular system of the patient further comprises the steps of:
ga. generating a series of correlation values, each correlation value associated with one of the pressure values, wherein each correlation value is an output of a correlation function with the body fluid volume indication value as a first input of the correlation function and the associated pressure value as a second input of the correlation function;
ha. defining a depletion correlation graph as comprising a first subset of the series of correlation values and associated pressure values, the first subset of the series of correlation values based on body fluid volume indication values obtained during depletion;
ia. defining a replenishment correlation graph as comprising a second subset of the series of correlation values and associated pressure values, the second subset of the series of correlation values based on body fluid volume indication values obtained during replenishment;
ja. determining a lowest depletion correlation value;
ka. determining a lowest replenishment correlation value; and
la. determining a hydration status of the patient based on the lowest depletion correlation value and the lowest replenishment correlation value.

18. The method of claim 6 wherein the step of identifying the relationship of the body fluid volume indication values referenced to the series of pressure values, thereby indicating the physiological parameter of the vascular system of the patient further comprises the steps of:
ma. generating a depletion slope graph by generating a series of volume vs pressure slope values based on the series of body fluid volume indication values during depletion, then graphing the series, of volume vs pressure slope values versus the series of pressure values;
na. identifying a maxima of the depletion slope graph as a first detection of depletion in one of the body fluid compartments of the vascular system of the patient; and
oa. identifying a minima of the depletion slope graph as a first detection of a mean pressure in the one of the body fluid compartments of the vascular system of the patient.

19. The method of claim 6 wherein the step of identifying the relationship of the body fluid volume indication values referenced to the series of pressure values, thereby indicating the physiological parameter of the vascular system of the patient further comprises the steps of:
pa. generating a replenishment slope graph by generating a series of volume vs pressure slope values, based on the series of body fluid volume indication values during replenishment, then graphing the series of volume vs pressure slope values versus the series of pressure values;
qa. identifying a minima of the replenishment slope graph with a first detection of filling in one of the body fluid compartments of the vascular system of the patient; and
ra. identifying a maxima of the replenishment slope graph with a first detection of mean pressure in the one of the body fluid compartments of the vascular system of the patient.

20. The method of claim 18, wherein the step of identifying the relationship of the body fluid volume indication values referenced to the series of pressure values, thereby indicating the physiological parameter of the vascular system of the patient further comprises the steps of:
sa. determining a stressed volume in the one the body fluid compartments based on a difference between a first volume and a second volume, wherein the first volume is associated with first detection of depletion in the one of the body fluid compartments and the second volume is associated with the first detection of mean pressure in the one of the body fluid compartments; and
ta. determining an unstressed volume in the one of the body fluid compartments based on a difference between a third volume and a fourth volume, wherein the third volume is associated with the first detection of mean pressure in the one of the body fluid compartments and the fourth volume is associated with a first detection of depletion in the next to be depleted body fluid compartment.

21. The method of claim 19, wherein the step of identifying the relationship of the body fluid volume indication values referenced to the series of pressure values, thereby indicating the physiological parameter of the vascular system of the patient further comprises the step of:
ua. determining a stressed volume in the one the body fluid compartments based on a difference between a first volume and a second volume, wherein the first volume is associated with the first detection of filling in a next replenished one of the body fluid compartments and the second volume is associated with the first detection of mean pressure in the one of the body fluid compartments; and
va determining an unstressed volume in the one of the body fluid compartments based on a difference between a third volume and a fourth volume, wherein the third volume is associated with the first detection of the mean pressure in the one of the body fluid compartments and the fourth volume is associated with a first detection of replenishment in the body fluid compartment.

22. The method of claims 20 or 21, wherein the step of identifying the relationship of the body fluid volume indication values referenced to the series of pressure values, thereby indicating the physiological parameter of the vascular system of the patient further comprises the step of:
wa. determining elastance in the body region based on a relationship of the stressed volume to the unstressed volume in the one of the body fluid compartments of the body region.

23. The method of claim 1, wherein the step of identifying the relationship of the body fluid volume indication values referenced to the series of pressure values, thereby indicating the physiological parameter of the vascular system of the patient further comprises the step of:
xa. determine arterial compliance based on the series of body fluid volume indication values and the series of pressure values;
ya. determine venous compliance based on the series of body fluid volume indication values and the series of pressure values; and
za. determining vascular status based on a ratio of arterial compliance to venous compliance.
